(19) United States
(12) Patent Application Publication
(10) Pub. No.: US 2025/0336063 A1
(43) Pub. Date: Oct. 30, 2025

(54) Multimodal Deep Learning to Differentiate Tumor Recurrence from Treatment Effect in Human Glioblastoma

(71) Applicant: University of Virginia Patent Foundation, Charlottesville, VA (US)

(72) Inventors: Bijoy Kundu, Charlottesville, VA (US); Zoraiz Qureshi, Framingham, MA (US); David Schiff, Charlottesville, VA (US); Thomas Muttikkal, Charlottesville, VA (US)

(21) Appl. No.: 19/064,678

(22) Filed: Feb. 26, 2025

Related U.S. Application Data
(60) Provisional application No. 63/557,699, filed on Feb. 26, 2024.

(51) Int. Cl. G06T 7/00; A61B 5/0275; G06T 7/12; G06T 7/30; G16H 30/20; G16H 30/40; G16H 50/20

(52) U.S. Cl. CPC G06T 7/0012; A61B 5/0275; G06T 7/12; G06T 7/30; G16H 30/20; G16H 30/40; G16H 50/20; G06T 2207/10088; G06T 2207/10104; G06T 2207/20081; G06T 2207/30096

(57) ABSTRACT

A computer implemented method of distinguishing tumor progression from treatment-related necrosis in digital images of a subject implements a multimodal deep learning architecture from MRI data of a selected anatomical portion of the subject, such as a brain, and corresponding dPET data for the subject with a tracer applied to the anatomical portion of the subject. The computer stores co-registered MRI data frames with dPET data frames as three dimensional (3D) parametric PET maps to identify multi-modal image features of segmented tumor data. A convolutional neural network uses MRI data and selected multi-modal image features as inputs and the computer concatenates respective output latent feature vectors from the respective sections of the at least one CNN. Feeding concatenated feature vectors to fully connected layers of the multimodal architecture distinguishes tumor progression from treatment-related necrosis of the anatomical portion of the subject.

### CROSS-REFERENCE TO RELATED APPLICATIONS

This application claims priority to and the benefit of U.S. provisional patent application No. 63/557,699, filed on Feb. 26, 2024, and titled Method for Optimizing Insulin Dosing Parameters in Computer Aided Dosing System, the disclosure of which is hereby incorporated by reference herein in its entirety.

### STATEMENT OF GOVERNMENT RIGHTS

None.

### BACKGROUND

In some embodiments, artificial intelligence and machine learning techniques may be used in optional embodiments of this disclosure. Machine Learning (ML) and Artificial Intelligence (AI) systems are in widespread use in customer service, marketing, and other industries, including medicine and science. Machine learning is considered a subset of more general artificial intelligence operations, and AI endeavors may utilize numerous instances of machine learning to make decisions, predict outputs, and perform human-like intelligent operations. Machine learning protocols typically involve programming a model that instantiates an appropriate algorithm for a given computing environment and training the model on a particular data set or domain with known historical results. The results are generally known outputs of many combinations of parameter values that the algorithm accesses during training. The model uses numerous statistical and mathematical operations to learn how to make logical decisions and generate new outputs based on the historical training data. Machine learning (ML) includes, but is not limited to, a number of models such as neural networks, deep learning algorithms, support vector machines, data clustering, regression models, and Monte Carlo simulations. Other models may utilize linear regression, logistic regression, support vector machines, K-means clustering, classification models such as a binary classifier or a multi-class classifier, clustering models, anomaly detection, other supervised learning models, and even combinations of one or more machine language model types. Most of these take vectors of data as inputs.

The term “artificial intelligence,” therefore, includes any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is generally a subset of AI that enables a machine to acquire knowledge by extracting patterns from raw data.

The term “representation learning” may be used as a subset of machine learning that enables a machine to automatically discover representations needed for feature detection, prediction, or classification from raw data. Representation learning techniques include, but are not limited to, autoencoders.

The term “deep learning” may also be considered a subset of machine learning that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns a function that maps an input (also known as feature or features) to an output (also known as target or target) during training with an unlabeled data set. In a semi-supervised model, the model learns a function that maps an input (also known as feature or features) to an output (also known as target or target) during training with both labeled and unlabeled data.

Some machine learning models are designed for a specific data set or domain and are highly expert at handling the nuances within that narrow domain. It is with respect to these and other considerations that the various aspects of the present disclosure as described below are presented.

This disclosure combines algorithms deciphered by artificial intelligence and machine learning with currently known systems and models that gather data from a patient on a real time basis. Accordingly this disclosure can utilize sensors and medical equipment that improve a system's ability to diagnose and treat a patient.

Brackets with numerals therein refer to references cited in this disclosure.

### SUMMARY

In an embodiment, a computer implemented method of distinguishing tumor progression from treatment-related necrosis in digital images of a subject includes using a computer having a processor connected to computer memory storing software that, when executed, performs computer instruction steps of a machine learning architecture by collecting magnetic resonance image (MRI) data of a selected anatomical portion of the subject and collecting dynamic positron emission tomography (dPET) data for the subject with a tracer applied to the anatomical portion of the subject. The MRI data frames and dPET data frames are co-registered and the computer stores a co-registered dPET volume of frames in the computer memory. Using the co-registered dPET volume, the method calculates and saves a tracer influx constant (Ki) map for the tracer at the anatomical portion of the subject. The method continues by segmenting respective tumor data from the MRI data and the Ki maps on a frame by frame basis and applying segmented MRI data and segmented Ki maps to respective sections of a dual encoder convolutional neural network (CNN). Concatenating respective output latent feature vectors from the respective sections of the dual encoder allows for feeding concatenated feature vectors to fully connected layers of the machine learning architecture to distinguish tumor progression from treatment-related necrosis of the anatomical portion.

In an embodiment, the method further includes injecting the tracer into the subject to model glucose transport to the anatomical portion of the subject.

In an embodiment, the method includes injecting Fluorine-18 fluorodeoxyglucose (18F-FDG) as a surrogate marker for glucose metabolism.

In an embodiment, the method includes training the CNN utilizing a supervised transfer learning procedure.

In an embodiment, the method includes storing three dimensional (3D) co-registered dPET tumor volumes in the computer memory.

In another implementation, a computer implemented method includes distinguishing tumor progression from treatment-related necrosis in digital images of a subject, and the method uses a computer having a processor connected to computer memory storing software that, when executed, performs computer instruction steps of a multimodal deep learning architecture to collect magnetic resonance image (MRI) data of a selected anatomical portion of the subject and further collect dynamic positron emission tomography (dPET) data for the subject with a tracer applied to the anatomical portion of the subject. The method continues by co-registering MRI data frames with dPET data frames and storing multi-channel parametric positron emission tomography (PET) volumes as three dimensional (3D) parametric PET maps in the computer memory. Using the 3D parametric PET maps, the method can identify and store multi-modal image features in the computer memory and continue by segmenting respective tumor data from both the MRI data and the 3D parametric PET maps on a frame by frame basis. The method includes applying segmented MRI data and selected multi-modal image features from the segmented 3D parametric PET maps to respective sections of at least one convolutional neural network (CNN) and concatenating respective output latent feature vectors from the respective sections of the at least one CNN. The method allows for feeding concatenated feature vectors to fully connected layers of the multimodal architecture to distinguish tumor progression from treatment-related necrosis of the anatomical portion of the subject.

In an embodiment, prior to storing the 3D parametric PET maps, the method includes performing a step of calculating an image derived blood input function (IDIF) that identifies an amount of tracer in the blood available for the anatomical portion to use.

In an embodiment, calculating the IDIF includes segmenting internal carotid arteries (ICA) of the subject from the 3D parametric PET maps co-registered with the MRI data frames.

In an embodiment, calculating the IDIF includes correcting the IDIF with multi-parameter modeling correcting for partial volume (PV) effects and spill over (SP) contamination to store a model corrected blood input function (MCIF) in the computer memory.

In an embodiment, the method further includes feeding the MCIF and the dPET data for the subject into a graphical Patlak model that performs a voxel-wise linear regression on the data to derive a rate of tracer uptake, Ki, as a slope.

In an embodiment, the method further utilizes the MCIF to compute voxel by voxel parametric maps of tracer kinetic rate constants and tracer influx constant.

In an embodiment, the method further includes convolving an ICA segmentation to compute an average blood time-activity curve across all time frames to produce a tracer time activity curve as an initial value for the IDIF.

In an embodiment, the method further includes the anatomical portion being a brain of a subject with a tumor therein, and the method further includes collecting radiomics features from respective voxels of the 3D parametric PET maps and/or radiomics from corresponding images of MRI tumor volumes, wherein the radiomics features comprise at least one of first-order statistics, 2D and 3D shape-descriptors, or texture level features.

In an embodiment, the method further includes concatenating respective output latent feature vectors further comprises adding to a concatenated feature vector with multimodal image features from the 3D parametric PET maps, wherein the multimodal features comprise metabolic uptake rate Ki, individual rate constants K1 to K3, total blood volume, tumor time-activity curves (TAC), or standardized uptake values (SUV).

In an embodiment, the method further includes collecting magnetic resonance image (MRI) data further comprises collecting multi-channel MRI data comprising T1 image data, T1c image data, t2/FLAIR image data, perfusion imaging, and diffusor tensor imaging (DTI).

In an embodiment, the method further includes concatenating respective output latent feature vectors further comprises adding to a concatenated feature vector with the multi-channel MRI data.

In an embodiment, the method further includes collecting static PET data for the anatomical portion of the subject and wherein concatenating respective output latent feature vectors further comprises the static PET data.

In an embodiment, the method further includes retrieving demographic data regarding the subject and wherein concatenating respective output latent feature vectors further includes the demographic data.

In another implementation, a system includes a computer having a processor connected to computer memory and in communication with an MRI imaging device and a PET scanning device, wherein the computer memory stores software that implements the multimodal deep learning architecture of this disclosure.

In an embodiment, the anatomical feature of the subject is a brain having a tumor.

### BRIEF DESCRIPTION OF THE DRAWINGS

Reference will now be made to the accompanying drawings, which are not necessarily drawn to scale. The patent or application file contains at least one drawing executed in color. Copies of this patent or patent application publication with color drawing(s) will be provided by the Office upon request and payment of the necessary fee.

FIG. 1A is a flow chart of an example method of utilizing machine learning processes to distinguish tumor progression from treatment related necrosis in a subject's anatomical portion such as a brain according to this disclosure.

FIG. 1B is a flow chart of an example method of utilizing a multimodal deep learning architecture to distinguish tumor progression from treatment related necrosis in a subject's anatomical portion such as a brain according to this disclosure.

FIG. 2 is a high level functional block diagram of an embodiment of the present disclosure, or an aspect of an embodiment of the present disclosure.

FIG. 3A is a computer architecture diagram showing a computing system capable of implementing aspects of the present disclosure in accordance with one or more embodiments.

FIG. 3B is a computer architecture diagram showing a networking environment that allows for data communication with a computing system capable of implementing aspects of the present disclosure in accordance with one or more embodiments.

FIG. 4 is a block diagram that illustrates a system 130 including a computer system 140 and the associated Internet 11 connection upon which an embodiment may be implemented.

FIG. 5 illustrates a system in which one or more embodiments of the disclosure can be implemented using a network, or portions of a network or computers. The present disclosure may be practiced with or without a network.

FIG. 6 illustrates an embodiment that includes, but is not limited thereto, a system, method, and computer readable medium that provides a binary classifier to determine if an image includes diseased tissue progression or treatment related necrosis.

FIG. 7A illustrates total intensity in a cropped region of a PET scan vs. scan timeframe, showing a rapid increase and subsequent plateau as the radiotracer accumulates in embodiments of this disclosure.

FIG. 7B illustrates total intensity differences with respect to a previous frame vs. scan time frame of a PET scan, identifying substantial changes in radiotracer presence and the ideal timeframe for segmentation.

FIG. 8 illustrates 3D-Unet based ICA-net [19] for segmentation of the ICA for automated derivation of IDIF.

FIG. 9A illustrates an ICA-net performance in terms of training and validation loss according to embodiments of this disclosure.

FIG. 9B illustrates an ICA-net performance in terms of training and validation DICE coefficient according to embodiments of this disclosure.

FIG. 9C illustrates an ICA-net performance in terms of training and validation Jaccard index according to embodiments of this disclosure.

FIG. 10 illustrates prediction results of ICA-net [19] and IDIF on two test subjects.

FIG. 11 illustrates RNN-based MCIF-net [19] for automated derivation of blood input with partial volume corrections according to embodiments of this disclosure.

FIG. 12A illustrates prediction results of ICA-net and IDIF on a first of two test subjects with a 3D visualization of Ground Truth GT of a first subject.

FIG. 12B illustrates prediction results of ICA-net and IDIF on a second of two test subjects with a 3D visualization of Ground Truth GT of a second subject.

FIG. 12C illustrates prediction results of ICA-net and IDIF on a first of two test subjects in MCIF graphs for the subjects with low MAE and MAPE values indicating accurate performance.

FIG. 12D illustrates prediction results of ICA-net and IDIF on a second of two test subjects in MCIF graphs for the subjects with low MAE and MAPE values indicating accurate performance.

FIG. 13A illustrates an end to end high level depiction of brain tumor classification [15] starting from data acquisition, motion correction, registration of 4D PET data in MR space, IDIF, MCIF and parametric brain PET map computation followed by brain tumor segmentation and classification.

FIG. 13B illustrates Parametric PET derivation and tumor voxel extraction [15] in SRI24 image space from Ki and MR voxels for classification.

FIG. 14 illustrates examples of processed tumor images from each modality (MR, SUV, Ki) used as network inputs.

FIG. 15 illustrates a proposed CNN architecture for ICA frame selection.

FIG. 16 illustrates a proposed UNETR architecture for ICA segmentation

FIG. 17 illustrates MCIF Prediction for test data with known surgical ground truth [17] including a comparison of original, ground truth, and UNETR predicted images alongside the MCIF prediction curve.

FIG. 18 illustrates a proposed cascade network architectures (UNETR/Transformer). A 3D UNETR based model can be trained to learn the structure and localization of the ICA for automated segmentation. Another sequence-sequence transformer-based network architecture for time-series regression using positional encoding can train and predict ground truth MCIF and downstream parametric Ki maps for test data using IDIF as input.

FIG. 19 illustrates proposed transfer learning and brain tumor segmentation pipeline for training on pre-surgical BraTS for post-treated brain tumor data.

FIG. 20 illustrates multimodal deep learning (MMDL) architecture for classification between tumor progression (TP) and treatment related necrosis (TN). The MMDL features include parametric PET, MR, radiomics and subject demographics.

FIG. 21A illustrates Table 1 as described herein.

FIG. 21B illustrates Table 2 as described herein.

FIG. 21C illustrates Table 3 as described herein.

FIG. 21D illustrates Table 4 as described herein.

### DETAILED DESCRIPTION

In some aspects, the disclosed technology relates to systems, methods, and computer-readable medium improving insulin therapy dosing. Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest.”

A detailed description of aspects of the disclosed technology, in accordance with various example embodiments, will now be provided with reference to the accompanying drawings. The drawings form a part hereof and show, by way of illustration, specific embodiments and examples. In referring to the drawings, like numerals represent like elements throughout the several figures.

An aspect of an embodiment of the present disclosure provides, among other things, a system, method and computer readable medium for providing deep learning methods and multimodal deep learning architectures to distinguish tumor progression in a patient's brain from tissue necrosis caused by tumor treatments.

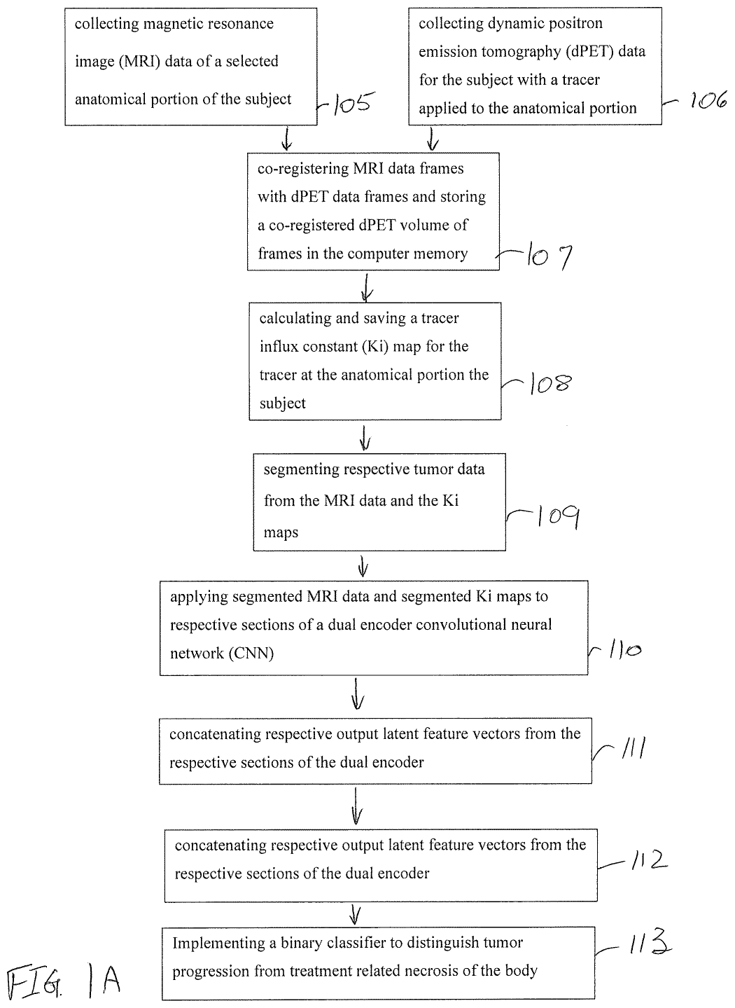

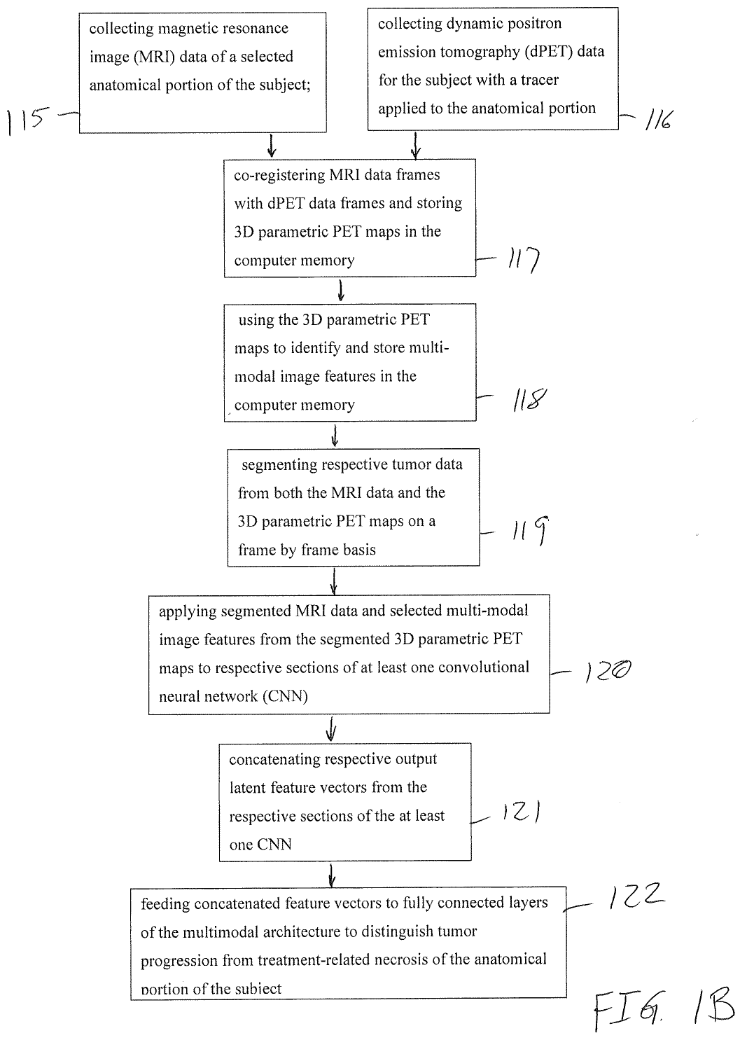

In an embodiment shown in FIG. 1A, a computer implemented method of distinguishing tumor progression from treatment-related necrosis in digital images of a subject includes using a computer having a processor connected to computer memory storing software that, when executed, performs computer instruction steps of a machine learning architecture by collecting 105 magnetic resonance image (MRI) data of a selected anatomical portion of the subject and collecting 106 dynamic positron emission tomography (dPET) data for the subject with a tracer applied to the anatomical portion of the subject. The MRI data frames and dPET data frames are co-registered 107 and the computer stores a co-registered dPET volume of frames in the computer memory. Using the co-registered dPET volume, the method calculates and saves a tracer influx constant (Ki) map 108 for the tracer at the anatomical portion of the subject. The method continues by segmenting 109 respective tumor data from the MRI data and the Ki maps on a frame by frame basis and applying segmented MRI data and segmented Ki maps to respective sections of a dual encoder convolutional neural network (CNN) 110. Concatenating respective output latent feature vectors from the respective sections of the dual encoder 111 allows for feeding concatenated feature vectors to fully connected layers 112 of the machine learning architecture to distinguish tumor progression from treatment-related necrosis of the anatomical portion 113.

In another implementation shown in FIG. 1B, a computer implemented method includes distinguishing tumor progression from treatment-related necrosis in digital images of a subject, and the method uses a computer having a processor connected to computer memory storing software that, when executed, performs computer instruction steps of a multimodal deep learning architecture to collect magnetic resonance image (MRI) data 115 of a selected anatomical portion of the subject and further collect dynamic positron emission tomography (dPET) data 116 for the subject with a tracer applied to the anatomical portion of the subject. The method continues by co-registering MRI data frames with dPET data frames 117 and storing multi-channel parametric positron emission tomography (PET) volumes as three dimensional (3D) parametric PET maps in the computer memory. Using the 3D parametric PET maps, the method can identify and store multi-modal image features 118 in the computer memory and continue by segmenting respective tumor data from both the MRI data and the 3D parametric PET maps on a frame by frame basis 119. The method includes applying segmented MRI data and selected multi-modal image features from the segmented 3D parametric PET maps to respective sections of at least one convolutional neural network (CNN) 120 and concatenating respective output latent feature vectors 121 from the respective sections of the at least one CNN. The method allows for feeding concatenated feature vectors to fully connected layers of the multimodal architecture to distinguish tumor progression from treatment-related necrosis of the anatomical portion of the subject 122.

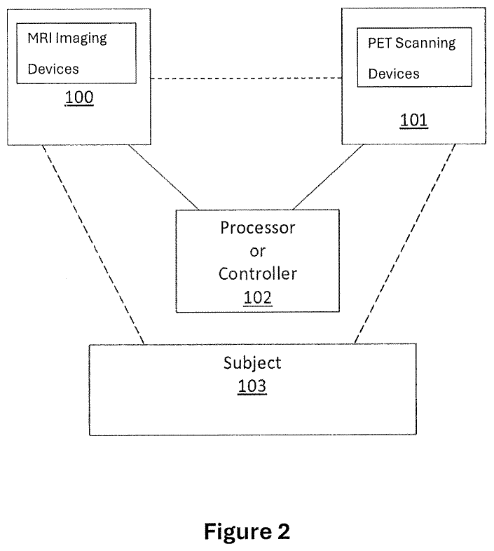

FIG. 2 is a high level functional block diagram of an embodiment of the present disclosure, or an aspect of an embodiment of the present disclosure.

As shown in FIG. 2, a processor or controller 102 communicates with the glucose monitor or device 101, and optionally the insulin device 100. The glucose monitor or device 101 communicates with the subject 103 to monitor glucose levels of the subject 103. The processor or controller 102 is configured to perform the required calculations. Optionally, the insulin device 100 communicates with the subject 103 to deliver insulin to the subject 103. The processor or controller 102 is configured to perform the required calculations. The glucose monitor 101 and the insulin device 100 may be implemented as a separate device or as a single device. The processor 102 can be implemented locally in the glucose monitor 101, the insulin device 100, or a standalone device (or in any combination of two or more of the glucose monitor, insulin device, or a stand along device). The processor 102 or a portion of the system can be located remotely such that the device is operated as a telemedicine device. FIG. 2 also illustrates sensors and detectors that can be used to gather field data measurements for a subject, in real time or from samples, from the patient's blood. These kinds of sensors and detectors may be stand alone equipment or incorporated into an insulin delivery device or pump.

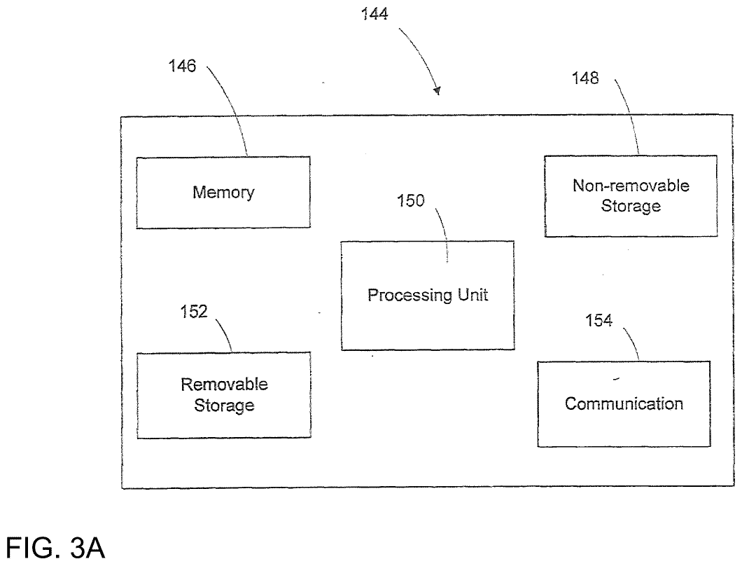

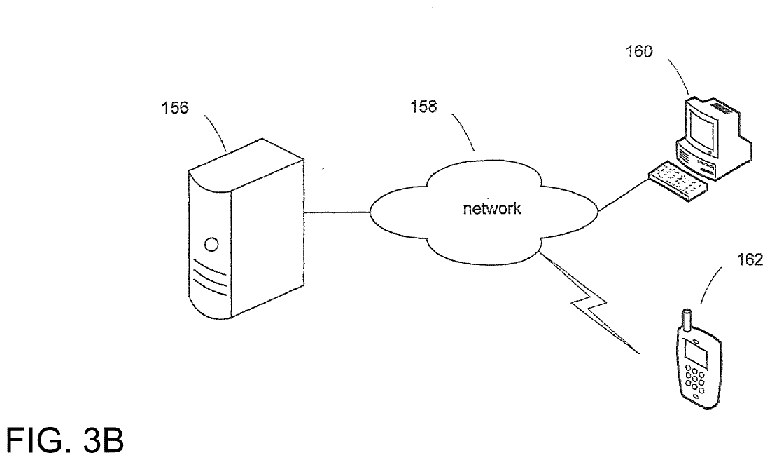

Referring to FIG. 3A, in its most basic configuration, computing device 144 typically includes at least one processing unit 150 and memory 146. Depending on the exact configuration and type of computing device, memory 146 can be volatile (such as RAM), non-volatile (such as ROM, flash memory, etc.) or some combination of the two.

Additionally, device 144 may also have other features and/or functionality. For example, the device could also include additional removable and/or non-removable storage including, but not limited to, magnetic or optical disks or tape, as well as writable electrical storage media. Such additional storage is the figure by removable storage 152 and non-removable storage 148. Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, the device.

The device may also contain one or more communications connections 154 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode, execute, or process information in the signal. By way of example, and not limitation, communication medium includes wired media such as a wired network or direct-wired connection, and wireless media such as radio, RF, infrared and other wireless media. As discussed above, the term computer readable media as used herein includes both storage media and communication media.

In addition to a stand-alone computing machine, embodiments of the disclosure can also be implemented on a network system comprising a plurality of computing devices that are in communication with a networking means, such as a network with an infrastructure or an ad hoc network. The network connection can be wired connections or wireless connections. As a way of example, FIG. 3B illustrates a network system in which embodiments of the disclosure can be implemented. In this example, the network system comprises computer 156 (e.g. a network server), network connection means 158 (e.g. wired and/or wireless connections), computer terminal 160, and PDA (e.g. a smart-phone) 162 (or other handheld or portable device, such as a cell phone, laptop computer, tablet computer, GPS receiver, mp3 player, handheld video player, pocket projector, etc. or handheld devices (or non portable devices) with combinations of such features). In an embodiment, it should be appreciated that the module listed as 156 may be glucose monitor device. In an embodiment, it should be appreciated that the module listed as 156 may be a glucose monitor device, artificial pancreas, and/or an insulin device (or other interventional or diagnostic device). Any of the components shown or discussed with FIG. 3B may be multiple in number. The embodiments of the disclosure can be implemented in anyone of the devices of the system. For example, execution of the instructions or other desired processing can be performed on the same computing device that is anyone of 156, 160, and 162. Alternatively, an embodiment of the disclosure can be performed on different computing devices of the network system. For example, certain desired or required processing or execution can be performed on one of the computing devices of the network (e.g. server 156 and/or glucose monitor device), whereas other processing and execution of the instruction can be performed at another computing device (e.g. terminal 160) of the network system, or vice versa. In fact, certain processing or execution can be performed at one computing device (e.g. server 156 and/or insulin device, artificial pancreas, or glucose monitor device (or other interventional or diagnostic device)); and the other processing or execution of the instructions can be performed at different computing devices that may or may not be networked. For example, the certain processing can be performed at terminal 160, while the other processing or instructions are passed to device 162 where the instructions are executed. This scenario may be of particular value especially when the PDA 162 device, for example, accesses to the network through computer terminal 160 (or an access point in an ad hoc network). For another example, software to be protected can be executed, encoded or processed with one or more embodiments of the disclosure. The processed, encoded or executed software can then be distributed to customers. The distribution can be in a form of storage media (e.g. disk) or electronic copy.

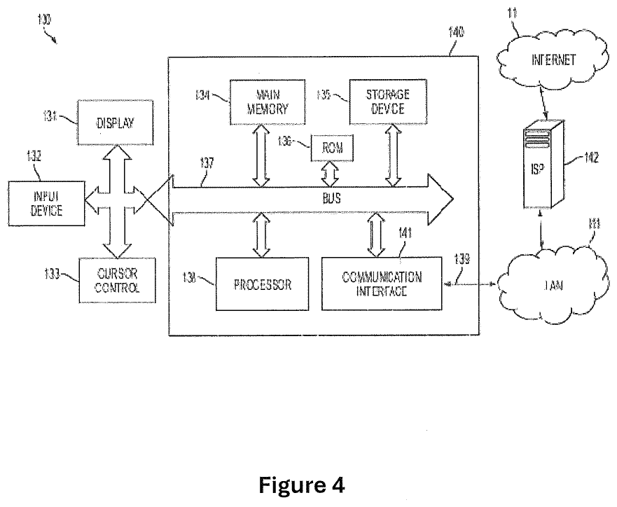

FIG. 4 is a block diagram that illustrates a system 130 including a computer system 140 and the associated Internet 11 connection upon which an embodiment may be implemented. Such configuration is typically used for computers (hosts) connected to the Internet 11 and executing a server or a client (or a combination) software. A source computer such as laptop, an ultimate destination computer and relay servers, for example, as well as any computer or processor described herein, may use the computer system configuration and the Internet connection shown in FIG. 4. The system 140 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 4 illustrates various components of a computer system, it is not intended to represent any particular architecture or manner of interconnecting the components; as such details are not germane to the present disclosure. It will also be appreciated that network computers, handheld computers, cell phones and other data processing systems which have fewer components or perhaps more components may also be used. The computer system of FIG. 4 may, for example, be an Apple Macintosh computer or Power Book, or an IBM compatible PC. Computer system 140 includes a bus 137, an interconnect, or other communication mechanism for communicating information, and a processor 138, commonly in the form of an integrated circuit, coupled with bus 137 for processing information and for executing the computer executable instructions. Computer system 140 also includes a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 further includes a Read Only Memory (ROM) 136 (or other non-volatile memory) or other static storage device coupled to bus 137 for storing static information and instructions for processor 138. A storage device 135, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically computer system 140 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 132, including alphanumeric and other keys, is coupled to bus 137 for communicating information and command selections to processor 138. Another type of user input device is cursor control 133, such as a mouse, a trackball, or cursor direction keys for communicating direction information and command selections to processor 138 and for controlling cursor movement on display 131. This input device typically has two degrees of freedom in two axes, a first axis (e.g., x) and a second axis (e.g., y), that allows the device to specify positions in a plane.

The computer system 140 may be used for implementing the methods and techniques described herein. According to one embodiment, those methods and techniques are performed by computer system 140 in response to processor 138 executing one or more sequences of one or more instructions contained in main memory 134. Such instructions may be read into main memory 134 from another computer-readable medium, such as storage device 135. Execution of the sequences of instructions contained in main memory 134 causes processor 138 to perform the process steps described herein. In alternative embodiments, hard-wired circuitry may be used in place of or in combination with software instructions to implement the arrangement. Thus, embodiments of the disclosure are not limited to any specific combination of hardware circuitry and software.

The term “computer-readable medium” (or “machine-readable medium”) as used herein is an extensible term that refers to any medium or any memory, that participates in providing instructions to a processor, (such as processor 138) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 137. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 138 for execution. For example, the instructions may initially be carried on a magnetic disk of a remote computer. The remote computer can load the instructions into its dynamic memory and send the instructions over a telephone line using a modem. A modem local to computer system 140 can receive the data on the telephone line and use an infra-red transmitter to convert the data to an infra-red signal. An infra-red detector can receive the data carried in the infra-red signal and appropriate circuitry can place the data on bus 137. Bus 137 carries the data to main memory 134, from which processor 138 retrieves and executes the instructions. The instructions received by main memory 134 may optionally be stored on storage device 135 either before or after execution by processor 138.

Computer system 140 also includes a communication interface 141 coupled to bus 137. Communication interface 141 provides a two-way data communication coupling to a network link 139 that is connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (June 1999), “Internetworking Technologies Handbook”, Chapter 7: “Ethernet Technologies”, pages 7-1 to 7-38, which is incorporated in its entirety for all purposes as if fully set forth herein. In such a case, the communication interface 141 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (Jan. 20, 2004), which is incorporated in its entirety for all purposes as if fully set forth herein.

Wireless links may also be implemented. In any such implementation, communication interface 141 sends and receives electrical, electromagnetic or optical signals that carry digital data streams representing various types of information. Network link 139 typically provides data communication through one or more networks to other data devices. For example, network link 139 may provide a connection through local network 111 to a host computer or to data equipment operated by an Internet Service Provider (ISP) 142. ISP 142 in turn provides data communication services through the world wide packet data communication network Internet 11. Local network 111 and Internet 11 both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

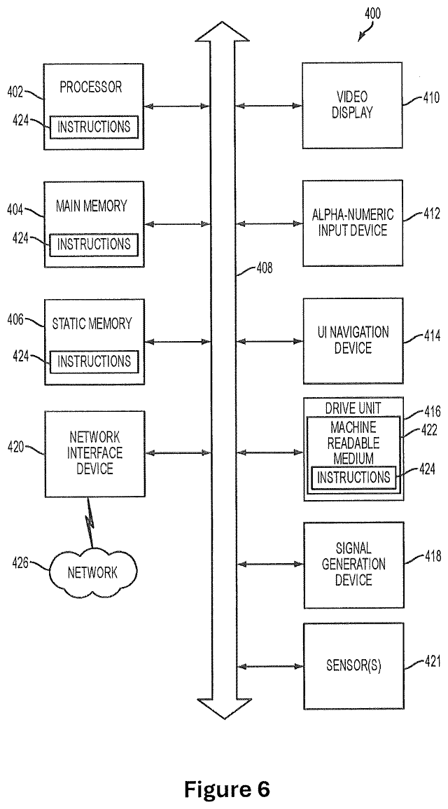

FIG. 6 is a block diagram illustrating an example of a machine upon which one or more aspects of embodiments of the present disclosure can be implemented.

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

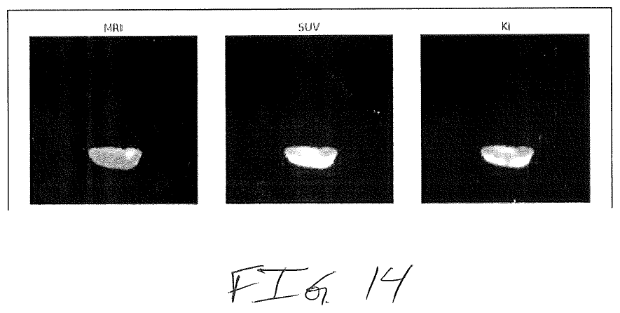

In an embodiment shown in FIG. 13A, a computer implemented method of distinguishing tumor progression from treatment-related necrosis in digital images of a subject includes using a computer having a processor connected to computer memory storing software that, when executed, performs computer instruction steps of a machine learning architecture 1300 by collecting magnetic resonance image (MRI) data 1305 of a selected anatomical portion of the subject and collecting dynamic positron emission tomography (dPET) data 1310 for the subject with a tracer applied to the anatomical portion of the subject. In a Ki map generation 1315, the MRI data frames and dPET data frames are co-registered 1318 and the computer stores a co-registered dPET volume of frames in the computer memory. Using the co-registered dPET volume, the method calculates and saves a tracer influx constant (Ki) map 1322 for the tracer at the anatomical portion of the subject. The method continues by segmenting respective tumor data from the MRI data 1326 and the Ki maps 1327 on a frame by frame basis and applying segmented MRI data and segmented Ki maps to respective sections 1328, 1329 of a dual encoder convolutional neural network (CNN). Concatenating 1330 respective output latent feature vectors from the respective sections of the dual encoder allows for feeding concatenated feature vectors to fully connected layers of the machine learning architecture to distinguish 1335 tumor progression from treatment-related necrosis of the anatomical portion.

In an embodiment, the method further includes injecting the tracer into the subject to model glucose transport to the anatomical portion of the subject.

In an embodiment, the method includes injecting Fluorine-18 fluorodeoxyglucose (18F-FDG) as a surrogate marker for glucose metabolism.

In an embodiment, the method includes training the CNN utilizing a supervised transfer learning procedure.

In an embodiment, the method includes storing three dimensional (3D) co-registered dPET tumor volumes in the computer memory.

In another implementation, a computer implemented method includes distinguishing tumor progression from treatment-related necrosis in digital images of a subject, and the method uses a computer having a processor connected to computer memory storing software that, when executed, performs computer instruction steps of a multimodal deep learning architecture to collect magnetic resonance image (MRI) data of a selected anatomical portion of the subject and further collect dynamic positron emission tomography (dPET) data for the subject with a tracer applied to the anatomical portion of the subject. The method continues by co-registering MRI data frames with dPET data frames and storing multi-channel parametric positron emission tomography (PET) volumes as three dimensional (3D) parametric PET maps in the computer memory. Using the 3D parametric PET maps, the method can identify and store multi-modal image features in the computer memory and continue by segmenting respective tumor data from both the MRI data and the 3D parametric PET maps on a frame by frame basis. The method includes applying segmented MRI data and selected multi-modal image features from the segmented 3D parametric PET maps to respective sections of at least one convolutional neural network (CNN) and concatenating respective output latent feature vectors from the respective sections of the at least one CNN. The method allows for feeding concatenated feature vectors to fully connected layers of the multimodal architecture to distinguish tumor progression from treatment-related necrosis of the anatomical portion of the subject.

In an embodiment, prior to storing the 3D parametric PET maps, the method includes performing a step of calculating an image derived blood input function (IDIF) 1320 that identifies an amount of tracer in the blood available for the anatomical portion to use.

In an embodiment, calculating the IDIF includes segmenting internal carotid arteries (ICA) of the subject from the 3D parametric PET maps co-registered with the MRI data frames.

In an embodiment, calculating the IDIF includes correcting 1321 the IDIF with multi-parameter modeling correcting for partial volume (PV) effects and spill over (SP) contamination to store a model corrected blood input function (MCIF) in the computer memory.

In an embodiment, the method further includes feeding the MCIF and the dPET data for the subject into a graphical Patlak model 1321 that performs a voxel-wise linear regression on the data to derive a rate of tracer uptake, Ki, as a slope.

In an embodiment, the method further utilizes the MCIF to compute voxel by voxel parametric maps of tracer kinetic rate constants and tracer influx constant.

In an embodiment, the method further includes convolving an ICA segmentation to compute an average blood time-activity curve across all time frames to produce a tracer time activity curve as an initial value for the IDIF.

In an embodiment, the method further includes the anatomical portion being a brain of a subject with a tumor therein, and the method further includes collecting radiomics features from respective voxels of the 3D parametric PET maps and/or radiomics from corresponding images of MRI tumor volumes, wherein the radiomics features comprise at least one of first-order statistics, 2D and 3D shape-descriptors, or texture level features.

In an embodiment, the method further includes concatenating respective output latent feature vectors further comprises adding to a concatenated feature vector with multimodal image features from the 3D parametric PET maps, wherein the multimodal features comprise metabolic uptake rate Ki, individual rate constants K1 to K3, total blood volume, tumor time-activity curves (TAC), or standardized uptake values (SUV).

In an embodiment, the method further includes collecting magnetic resonance image (MRI) data further comprises collecting multi-channel MRI data comprising T1 image data, T1c image data, t2/FLAIR image data, perfusion imaging, and diffusor tensor imaging (DTI).

In an embodiment, the method further includes concatenating respective output latent feature vectors further comprises adding to a concatenated feature vector with the multi-channel MRI data.

In an embodiment, the method further includes collecting static PET data for the anatomical portion of the subject and wherein concatenating respective output latent feature vectors further comprises the static PET data.

In an embodiment, the method further includes retrieving demographic data regarding the subject and wherein concatenating respective output latent feature vectors further includes the demographic data.

In another implementation, a system includes a computer having a processor connected to computer memory and in communication with an MRI imaging device and a PET scanning device, wherein the computer memory stores software that implements the multimodal deep learning architecture of this disclosure.

In an embodiment, the anatomical feature of the subject is a brain having a tumor.

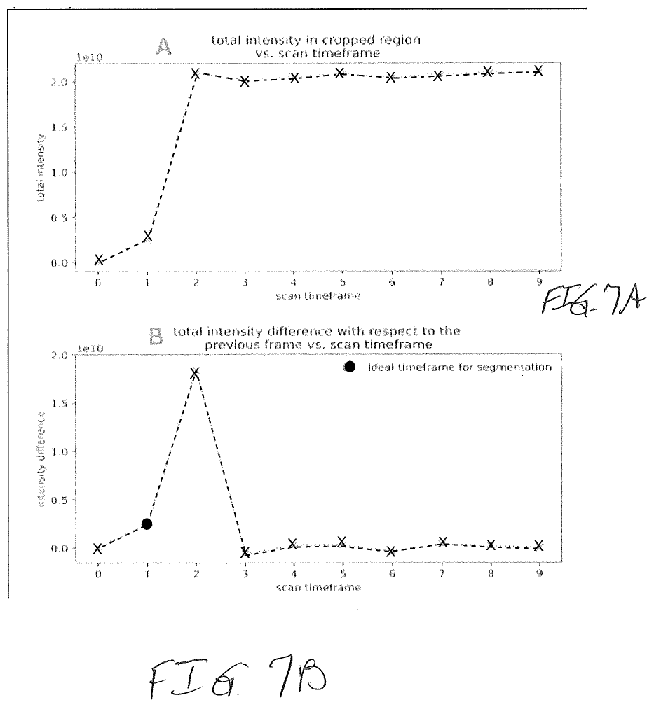

To specifically ensure that the model only focuses on tumor voxels instead of brain background voxels, we perform a semi-automated segmentation of tumor volumes using the seed-based region growing segmentation tool in 3D Slicer. This is followed by Gaussian smoothing to get a conservative mask of the abnormality and then verification from clinical experts. These masks are dropped onto co-registered PET Ki, SUV, and Ti-weighted MRI images to mask out the tumor. 3D parametric PET Ki, static PET standardized uptake value (SUV), and MR tumor voxels are extracted in the same image space. To perform the extraction of 3D tumor voxels while consistently preserving positional information, as a pre-processing step, the images and tumor mask for each subject are co-registered to the SRI24 atlas [6] with dimensions (240, 240, 155) and re-oriented into Left Posterior Superior orientation, This is done by registering the MR to the atlas after performing temporary N4 bias field correction and mutual information for rigid registration using the Cancer imaging Phenomics Toolkit (CaP11) [7], then using the computed transformation to bring the other modalities to the same space after which the mask is applied. The masked images were further center cropped to the brain to (170,170,1120) dimensions by computing a minimum viable bounding box across all datasets to remove unnecessary background voxels and bring focus to the tumors. In FIG. 8, we visualize the extracted tumor voxels for each modality. We observe a higher signal-to-noise ratio showcasing distinct tumor metabolic features within the Ki maps compared to SUVs.

High grade gliomas (HGGs) (World Health Organization [WHO] grade III and IV) are diffusely infiltrative primary brain neoplasms. Glioblastoma (GBM) accounts for 52 percent of all malignant primary brain neoplasms and has an incidence of 3 per 100,000 adults per year [1]. Surgical resection and adjuvant radiotherapy and chemotherapy are the mainstay of disease management, but there are no curative therapies [2]. HGGs invariably recur, appearing on conventional magnetic resonance imaging (MRI) as new/enlarging contrast-enhancing lesions or hyperintense signal abnormalities on T2/FLAIR sequences [2, 3]. Confusingly, brain tissue changes induced by chemoradiotherapy (“treatment effect”) commonly produce a similar MRI appearance. As such, distinguishing recurrent neoplasm (tumor progression (TP)) from treatment effect (tumor necrosis (TN)) is critical for clinical management decisions. Radiosurgery has become a primary treatment modality, but often results in brain tissue changes that mimic recurrence on conventional MRI. Dynamic FDG PET (dPET), an advance from traditional static FDG PET, may prove advantageous in clinical staging [4, 5].

Preliminary studies of dPET imaging of 26 subjects with GBM obtained using the whole-body time of flight (TOF) Siemens Biograph PET scanner6 at the University of Virginia (UVA) are very encouraging in the classification of treatment effect from recurrent neoplasm in GBM patients [7]. T1 weighed MPRAGE MRI was also performed on the same subjects using the Siemens 3T MR. For modeling of dPET data and delineation of tumor boundaries on modeled parametric PET maps, motion correction and co-registration processes of dPET volumes in MR space were designed using software from the FMRIB's Software Library (FSL) tool kit [8-12]. One of the key factors in modeling of dPET data is image derived blood input function (IDIF), which is the amount of tracer in the blood available for the tissue to use. Recent work from our lab [13] developed a formalism for computing model corrected blood input function (MCIF) for human dynamic brain FDG PET imaging from IDIF [4, 5]. The MCIF computation involves algorithmic selection and manual annotations of the internal carotid arteries (ICA) to measure IDIF followed by multi-parameter modeling correcting for partial volume (PV) effects and spill over (SP) contamination. The modeled MCIF was then utilized to compute whole brain voxel by voxel parametric maps of FDG kinetic rate constants and hence net FDG influx constant (Ki) in a 3-compartment glucose transport model [14].

A shallow 3-layer convolution neural network (CNN) [15-17] was developed and trained for classification be-tween TP and TN for 35 brain tumors (2 classes; 26 TPs/1 and 9 TNs/0; labeled by imaging follow up plus surgical pathology in some cases) in the PET-MR image space. Adjustments for class imbalance were performed by using weighted categorical cross-entropy based on the training distribution of labels (TN:TP balanced class weight ratios 1.95:0.65). 3D parametric PET Ki, static PET standardized uptake values (SUV) and also the brain tumor MR voxels formed the input for the CNN. The average test accuracy across all leave-one-out cross-validation iterations was 0.56 using only the MR, 0.65 using only the SUV, and 0.71 using only the Ki voxels (single channel CNN). Combining SUV and MR voxels in a dual channel/dual encoder CNN increased the test accuracy to 0.62. On the other hand, MR and Ki voxels increased the test accuracy to 0.7415. Thus, parametric PET Ki features alone or with MR features in deep learning models would enhance prediction accuracy in differentiating TP vs TN in GBM. Additionally, parametric PET Ki which models glucose transport into the tumors with underlying corrections for blood input is superior to static PET SUV. We hypothesize that deep learning based automatic selection and segmentation of the 3D ICA and MCIF prediction with PV corrections (for efficient and precise parametric PET maps) [18, 19], efficient transfer-learning enabled segmentation of brain tumors and multi-modal learning including tumor radiomics and patient demographics may improve classification accuracy with a higher precision in post treated GBM patients. This is expected to result in improved patient outcomes by more accurately tailored treatments. To test this hypothesis, this disclosure will pursue the following specific aims:

Aim 1. Development of algorithms for ICA frame selection, segmentation and MCIF prediction. Recent work from a lab developed a 3D-Unet based ‘ICA-net’ and RNN based ‘MCIF-net’ for ICA segmentation and derivation of blood input with PV corrections, respectively, for automated parametric FDG brain PET mapping [19]. A custom 3D-CNN framework for automated frame selection and UNETR for ICA segmentation to compute IDIF will be developed [18]. A cascade UNETR [20] and sequence to sequence transformer networks [21] will be developed to map IDIF to reference modeled MCIF (ground truth) computed using dual-output FDG transport model [4, 5].

Aim 2: Automated segmentation and classification of brain tumors for clinical validation. N=100 dynamic FDG brain PET and MR imaging of post treated GBM subjects will be performed over a period of 5 years. Transfer learning22 from MR based pre-treated BraTS (Brain Tumor Segmentation)23, 24 whole tumor segmentations will be developed for automated segmentation of our post-treated whole tumor MR data sets and compared to manual or semi-automated segmentations (ground truth) [7, 15]. A new and efficient 3D multimodal deep learning architecture 25 for classification between TP and TN will be developed. The multimodal information will include multi-channel parametric PET (kinetic, K1-k3; blood volume, TBV maps) and MRI (T1, T1c, T2/FLAIR, perfusion, DTI), static PET (all tumor voxels); tumor dPET standardized uptake values time activity curves, tumor radiomics [26, 27] and patient demographics. The prediction accuracy will test against surgical pathology (ground truth).

Glioblastoma (GBM) is a highly aggressive brain neoplasm with a median survival of 15 months [1]. Surgical re-section and adjuvant radiotherapy and chemotherapy are palliative rather than curative [2]. One barrier to treatment is that brain tissue changes induced by chemoradiotherapy commonly produce similar neuroimaging changes to tumor recurrence [2, 3]. As such, distinguishing recurrent neoplasm (tumor progression (TP)) from treatment effect (tumor necrosis (TN)) is critical for clinical management decisions [28]. Advanced MRI techniques [29-31] including diffusion [32, 33] and perfusion imaging [33-36] have yielded inconsistent and unreliable results for differentiating between these entities. Positron emission tomography (PET) with Fluorine-18 fluorodeoxyglucose (18F-FDG) serving as a surrogate marker for glucose metabolism, represents an imaging technique that can provide pathophysiologic and diagnostic data in this clinical setting [37]. Qualitative visual analysis by performing comparisons be-tween pathologic and normal appearing brain regions [38, 39] and standardized uptake values (SUV) measured at a specific time point post FDG injection has been widely used as a semi-quantitative measure but does not reliably differentiate tumor from post-therapy changes in the standard static PET imaging protocol [40]. Chen et al evaluated the diagnostic accuracy of dynamic 18F-FDOPA PET in imaging brain tumors in comparison to static FDG using standard of care SUV analysis [41]. Recent work by Wardak et al [42] in a dynamic PET study indicated the combined use of FLT and FDOPA data in predicting the overall survival of patients with recurrent brain tumors using multiple linear regression analysis. In another study by Dunet et al [43], the performance of 18F-FET was compared to FDG for the diagnosis and grading of brain tumors. The authors conclude that FET-PET per-formed better than FDG especially in assessing a new isolated brain tumor. Although most studies indicate that the studied radiotracers offer benefits over the widely used modality (FDG-PET), direct comparisons among the various amino acid tracers are limited, and none of them appear to have a clear advantage over the others in all aspects of brain tumor imaging [44]. In addition, none of these studies utilized the combined utility of para-metric FDG PET and AI in the differential diagnosis of recurrent vs necrotic tumors.

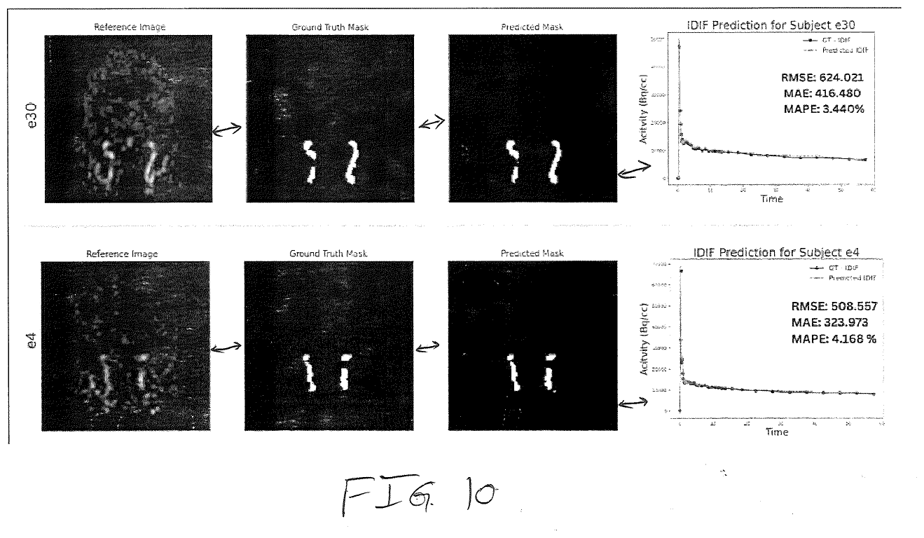

AI pipeline for parametric FDG brain PET mapping. Dynamic PET (dPET) measures radiation over a series of time windows for about 60 minutes; analysis performs convolution of the 4D brain PET data with blood input in a dual output kinetic model to form a 3D parametric brain PET map, which according to pilot studies in the Kundu lab at UVA, provides meaningful information not available from standard static PET [4, 5, 15, 16, 45]. Clinical adoption is still a challenge however due to difficult analysis protocols especially computation of blood input. Prior works performed model based blood input derivation utilizing local means analysis (LMA) as described [46]. Recent work from our lab [47] and others [48] automatically derived blood input using a LSTM network with IDIF as input for dynamic FDG PET images of mouse hearts. Development of a turn-key end-to-end AI powered dPET pipeline including blood input compute with partial volume corrections will be a significant step for automated FDG human brain PET mapping. Our new development [19] (BPEX 2024) is directly from dynamic human brain PET data as opposed to other works which utilize MRI [49] and CT based land-marking techniques [50] for blood input compute for whole-body dynamic PET images. An important step in this pipeline is the development of automatic frame selector from 4D-PET data for identification of the internal carotid arteries (ICA). Recent work from the Kundu lab developed an algorithm for automatic selection of the ICA frame for segmentation to derive IDIF 19. Another work by Moradi et al 51 combined wavelets and unsupervised learning for isolating arterial IDIF. New preliminary work from our lab developed a 3D convolution neural network (CNN) 18 for automatic ICA frame selection (proposed architecture, FIG. 10) which is a significant improvement over algorithmic selection of ICA (86% 18 vs 69% 19). The model proposed by Moradi et al 51 is far simpler as it identifies curves that have large peaks and small tails, while we let the CNN automatically identify patterns. The authors compare their methods with ground truth image-derived IDIF generated from the descending aorta without accounting for partial volume averaging (PV) and cardiac and respiratory motion. Spill over and PV effects would severely confound ground truth IDIF and hence validity of the computed arterial curves. The end to end AI-driven pipeline will identify the ICA, segment it and perform PV corrections accounting for spill-over contamination from surrounding voxels automatically. The development and retraining of a 3D-convolution neural network (CNN) model will be a significant step for automated brain PET mapping (Aim 1). Recent works from the Kundu lab improved on the shallow 3D Unet and a recurrent neural network (RNN; long short term memory network (LSTM)) with a Unet with transformer layers (UNETR) and a combination of LSTM and gated recurrent unit (GRU) frameworks for automatic segmentation of ICA and derivation of blood input with partial volume (PV) corrections [18]. Developing and training a cascade UNETR for ICA segmentation and sequence-sequence transformer for blood in-put compute will be significant for capturing long-range dependencies and variances in ICA structures across subjects and transfer learning to other tracers (Aim 1).

CNN for brain tumor classification. Preliminary work from our lab developed a custom dual encoder shallow convolution neural network (CNN) 15 and trained on 3D tumor PET Patlak Ki and MR voxels to classify TP vs TN. Although MRI is the standard of care in training deep learning models, utilizing MR and Ki voxels increased the test accuracy to 0.74 (compared to 0.56 for MR only) 15. Dynamic PET information, which models glucose influx into the tumors with underlying corrections for PV averaging of blood input, thus increased prediction accuracy. Prior works have explored radiomics feature extraction from multi-modal MRI 52 and diffusion MRI 53 along with feature selection and oversampling methods for this task but yield limited accuracy and have not evaluated the combination of metabolic image modalities such as PET with these structural ones. Another work follows the same input function derivation methodologies to compute average tumor Ki and other kinetic rate constants for classification using linear regression models 7. However, in comparison, not only do we utilize image features directly and develop more complex deep learning-based CNN approach, which can scale better with more data, but also utilize multi-modal combinations involving MRI and perform evaluation against static PET SUV maps for classification. Increasing the sample size for efficient utility of transfer learning 22 for brain tumor segmentation and 3D multimodal transformer based architecture25 including tumor radiomics 26, 27 and patient demographics (Aim 2) will be significant for efficient, accurate and precise classification of TP vs TN.

Impact: Improving the accuracy of deep learning algorithms to evaluate post-treatment patients with brain malignancies should improve patient management, and result in more accurate treatment decisions and ultimately in better patient outcomes.

One overarching goal of this disclosure is to enable improved classification of tumor progression from treatment effect (TP vs TN) using machine learning in human GBM utilizing multi-channel parametric PET for the first time in combination with MRI in the laboratory.

1) Frame selector (coined frame-Net): A 3D-CNN for automated frame selection will be developed and re-trained for the first time which will eliminate manual or algorithmic selection of early time frames for ICA localization.

2) ICA segmentation (coined ICA-Net): This will eliminate the need for arterial blood sampling for derivation of blood input for dynamic brain PET imaging. Existing technologies for segmentation of ICA from dynamic brain PET data is manual for model based 4, 5 derivation of image derived blood input function (IDIF). Recent new work 19 from our lab developed a 3D U-Net for automatic segmentation of ICA and IDIF derivation (see prelim data) 54, 55. UNETR developed and retrained for the first time for dynamic PET will encode global multi-scale context better yielding improved ICA segmentation performance compared to U-Net which has limited capability of learning long-range dependencies. This will remove bias (when compared to limited manual 2D slices for IDIF derivation) and inter-user dependence for consistent and efficient derivation of IDIF for the first time.

3) MCIF derivation (coined MCIF-Net): We will for the first time develop an approach for sequence-sequence transformer based model corrected blood input function (MCIF) with partial volume and spill over corrections from UNETR derived IDIF. Prior work from our lab developed a RNN (LSTM) [56, 57] based approach from total body parametric PET images in rodents for automatic derivation of MCIF [47]. Recent new work developed and adapted the RNN (LSTM plus bi-directional GRU) [19] to map IDIF to MCIF for parametric human FDG brain PET 54. One disadvantage of LSTM is that it is difficult to transfer learn and that the only layer we can tune is the final dense layer and not the LSTM parameters [58]. Developing sequence to sequence transformers for MCIF derivation for the first time will help with transfer learning e.g. to other tracers as well. This will allow for consistent and efficient derivation of downstream parametric PET maps.

4) Tumor segmentation and multimodal deep learning based classification (MMDL): This is the first attempt at an end-to-end approach for automated segmentation using transfer learning and multimodal classification (TP vs TN) of primary brain tumors in human GBM combining multi-channel parametric PET, multi-parametric MRI, tumor radiomics and subject demographics

Preliminary Data [19]. In the following this disclosure provides preliminary data for the development of an AI platform for blood input computed with PV corrections for dynamic FDG brain PET. This includes a 3D-Unet based ICA-net and RNN based MCIF-net for ICA segmentation and blood input compute with PV corrections respectively. Next, this disclosure provides preliminary data for brain tumor classification utilizing the recently developed multi-channel multi-encoder CNN framework including the parametric PET Ki, MR and traditional SUV voxels in image space.

The parametric brain PET pipeline comprises of four distinct phases (BPEX 2024) [19]. The first phase encompasses data preprocessing, which includes motion correction and co-registration of the dynamic FDG-PET (dFDG-PET) data. The second phase utilizes the 4D motion-corrected and co-registered data for internal carotid arteries (ICA) segmentation through ICA-net. The third phase involves the computation of the Image-Derived Blood Input Function (IDIF), monitoring changes in intensity within the ICA region. This phase also includes the application of MCIF-net for correcting PV and SP errors, resulting in the final MCIF. The fourth phase employs the MCIF to calculate the parametric brain PET map using a graphical Patlak model.

Data acquisition and preprocessing [4, 5, 19, 45, 60, 61]: The dFDG-PET imaging was conducted on a cohort comprising of 50 participants using the Siemens Biograph time-of-flight mCT scanner. The scans featured a resolution of 400 pixels×400 pixels×110 slices×38 time frames, and were conducted with time-dependent attenuation correction. The dynamic acquisition process involved the initiation of a 60-minute scan followed im-mediately with an intravenous administration of approximately 10 mCi FDG, injected over a duration of 10 seconds. All data were reconstructed in a list-mode format. Prior to the dFDG-PET procedure, all 50 patients under-went T1-weighted MPRAGE MPI (256 pixels×256 pixels×192 slices) using a Siemens 3T scanner for co-registration and anatomic mapping. The initial step in pre-processing started with motion correction for the 60-minute acquisition to align and lock the anatomy in the same 3 dimensional space throughout the entire time period as described [5, 60, 62]. Briefly, PET data (400 pixels×400 pixels×111 slices×38-time frames) was averaged across the first 14 frames to create a reference used to perform a rigid body transform across the 38 frames. The average of all the motion corrected PET frames were resliced and co-registered with T1weighted MRI using non-rigid transform to generate a transformation matrix used, in turn, to generate a co-registered dynamic PET volume. All the above registration processes were designed using software from the FMRIB's Software Library (FSL) tool kit [8-12].

Frame selector for 4D PET data set [19] (FIG. 7). This first step of this algorithm was to perform a center crop of the first frame based on the central position of the carotids with respect to the neck and brain. Next, the algorithm calculated the sum of intensity across all voxels in the cropped region. These two steps were then performed iteratively across the first 10 frames, generating a plot of total intensity as a function of scan timeframe (FIG. 7A). From this discrete function, the difference between each frame's summed intensity and the previous frame's summed intensity was calculated, generating a plot of summed intensity difference with respect to the previous frame as a function of scan timeframe (with the first frame having an intensity difference of its original summed intensity value) (FIG. 7B). The total intensity rapidly increases across the first several frames as the radiotracer is transported to the brain and then distinctly plateaus once the majority of the radiotracer has accumulated in the region. By plotting the intensity difference with respect to the previous frame, we can programmatically determine where substantial changes in radiotracer presence occur. With these differences, the algorithm then selected the frame that occurred one frame before the first local maximum. This local maximum indicated a large radiotracer presence distributed throughout the brain, meaning that the majority of the radiotracer had already been transported through the carotids. The previous frame, there-fore, represented the frame in which a considerable amount of radiotracer was still being transported through the carotids and the arteries were, thus, most clear for segmentation. All incorrectly selected frames were no more than 1 frame off. The accuracy of this method was 65.71%.

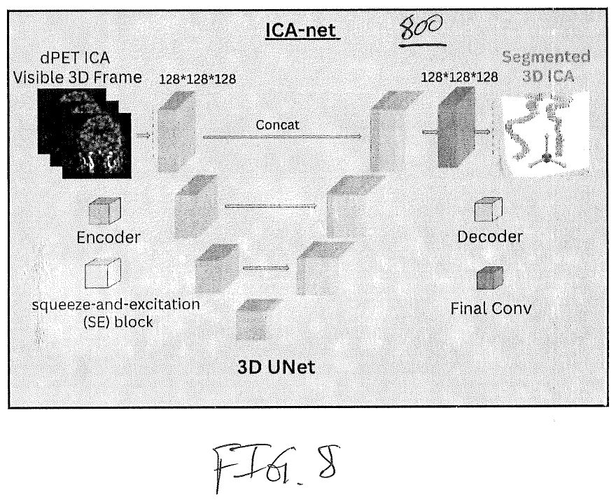

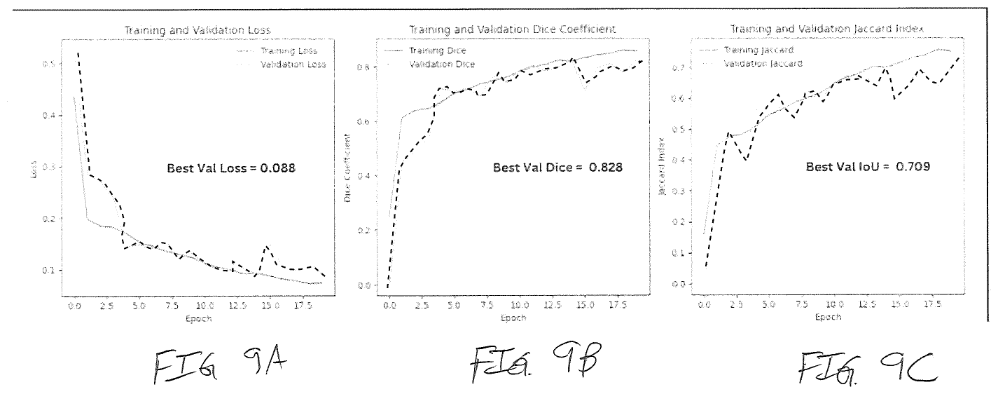

ICA-net [19, 54, 55]. The ICA-net (FIG. 8) is a customized model designed for the segmentation of ICA in 3D dFDG-PET scans. The ICA-net model leveraged a modified 3D U-Net architecture [63], specifically adapted for handling the unique challenges presented by dFDG-PET data. The preprocessing of ICA annotations, derived from a semi-automated segmentation method using 3D slicer, included binarization of the volume data. This was achieved through a custom ‘binarize Volume’ function, which applied a threshold to convert the scan data into a binary format, thereby simplifying the learning process for the ICA-net seg-mentation model. ICA-net's U-Net architecture was built on the backbone of VGG [16]. The input to the network was a 3D volume of shape 128×128×128, with a single channel representing grayscale images. Choosing the number of layers and nodes in this U-Net model involves balancing computational resources and model performance. In our model, we start with 64 filters in the first convolutional layer and double the number of filters at each subsequent down sampling step, up to 512 filters. This progressive increase in filters allows the model to learn more complex features at deeper layers. The depth of the model, with a total of five down sampling and up sampling stages, is designed to capture a wide range of spatial hierarchies. This design choice is guided by empirical testing and the specific requirements of the segmentation task. By leveraging convolutional layers, max-pooling, and up sampling operations, our model effectively reduces dimensionality and captures high-level features before reconstructing the image, ensuring precise and detailed segmentation outputs. Additionally, we have incorporated Squeeze-and-Excitation (SE) blocks into our model. These blocks help to recalibrate feature maps by channel-wise weighting, improving the model's sensitivity to important features and enhancing overall performance. The network was trained using a combined loss function consisting of Dice loss and binary cross-entropy (BCE) loss. The combined Dice and BCE Loss function integrated the ad-vantages of both Dice and BCE loss functions to address ICA segmentation challenges including class imbalance compensation, gradient behavior, comprehensive error signal, optimization landscape and robustness. This combined loss function facilitated im-proved model convergence and generalization (FIG. 9). Furthermore, the ICA-net employed several advanced techniques to bolster its performance and robust-ness. Data augmentation 64 plays a pivotal role, wherein each 3D PET brain image was trans-formed through randomized rotations (up to ±4 degrees), shifts (up to 5 voxels), and zooms (±0.05 scale factor), subsequently adjusted to a uniform size of 128×128×128 voxels through zero-intensity padding and cropping. This process effectively expanded the training dataset, enhancing the model's exposure to diverse clinical scenarios. Additionally, a batch size of 1 and the Adam optimizer for 120 epochs was utilized for efficient network training with its adaptive learning rate capabilities. Utilizing 5-fold cross validation, the model also implemented early stopping and model checkpointing strategies to prevent overfitting and to retain the best model weights during training. Finally, the model's parameters, totaling over 71 million, underscored its complexity and capacity to capture detailed features essential for accurate ICA segmentation. The validation metrics for ICA-net revealed a dice similarity score of 0.82 and a Jaccard score of 0.69 indicating the networks ability to learn a complex structure such as the ICA (Table 1). FIG. 10 illustrates the segmented results, where it is distinctly observable how the predicted ICA closely aligns with the original ICA, demonstrating the model's precision in segmentation and IDIF derivation.

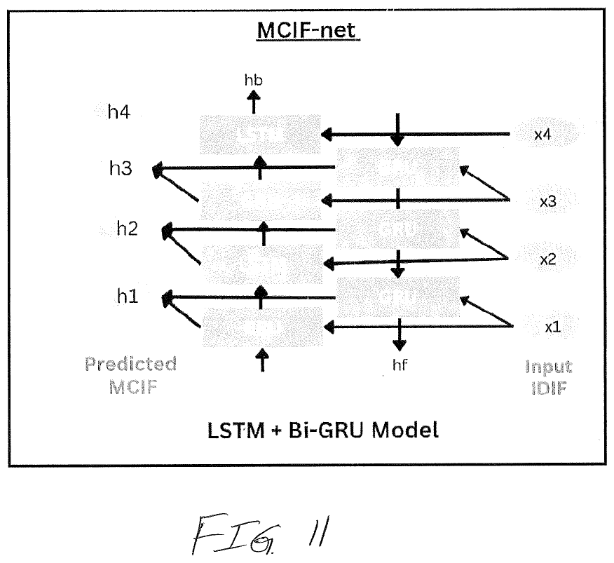

MCIF-net [19, 54]. (FIG. 11). To generate the parametric brain PET maps, reference modeled MCIF (ground truth) was computed by optimizing the IDIF derived from the ICA to account for partial volume recovery of the blood input [5]. The MCIF-net model was developed using a hybrid recur-rent neural network architecture, specifically designed to handle the time-series data inherent in dynamic PET imaging. This architecture integrated both LSTM and Bi-directional GRU layers [65], leveraging the strengths of each to enhance the processing of PET scan data. The LSTM layers, with increasing complexity from 50 to 200 neurons, were adept at capturing long-term dependencies, while the inclusion of Bi-directional GRU layers provided a richer context by processing data in both forward and backward directions. Dropout layers were interspersed throughout to pre-vent overfitting. Time-Distributed Dense layers were incorporated to maintain the temporal structure of the output, aligning with the sequential nature of the input. Data preparation for this combined architecture involved reshaping it into a format suitable for time-series analysis, specifically [samples, time steps, features]. The data was split according 5-fold cross validation. Compiled with the Adam optimizer and trained on a mean squared error loss function, MCIF-net underwent fine-tuning over 1000 epochs with a batch size of 32. With a total of 5,127,801 trainable parameters, the model's sophisticated architecture was strategically designed to maximize accuracy in predicting MCIF, a critical factor in PET scan analysis. The performance metrics (MAE and MSE) for the different architectures employed for model training indicated that a combination of Bi-directional GRU+LSTM outperformed LSTM, GRU and Bi-directional GRU alone (Table 2). Example 3D ICA and predicted MCIF compared to the ground truth segmentations and MCIF (model) respectively, are illustrated for two example datasets in FIG. 6. In the figure MAE and MAPE metrics were computed using the activity scale (Bq/cc). Table 3 presents the results of the 5-fold cross-validation, where the MAE, MSE, and RMSE metrics have been scaled or normalized between 0 and 1, as captured during model training. In Table 3, we have additionally computed the downstream Ki values, denoted as “Abs Ki Diff.” for two ground truth test subjects for each fold. This demonstrates the variance in the Ki map generated by our efficient deep learning model (MCIF-net), in comparison to our previous approach. Additionally, we also demonstrate that just using IDIF as opposed to MCIF in model computations results in larger average differences across 5-folds between model and predicted Ki maps (0.0056 vs 0.0009). Performance of both the networks (ICA-net and MCIF-net) in this work are an improvement over our previous work [54, 55] due to improved networks and an increase in data sets for model training.

Brain tumor classification 13, 20 (FIG. 7) Dynamic FDG PET scan of the brain was performed on 26 GBM patients using the Siemens Biograph time of flight (TOF) mCT scanner 6 to produce a DICOM file that contained a complete four dimensional image of each patient's brain tracer update over time. Dynamic acquisition consisted of an intravenous ˜10 mCi tracer injection over 10 seconds with initiation of a 60-minute scan in list-mode format. PET was preceded by a high resolution post-contrast T1-weighted MPRAGE MRI (256 pixels×256 pixels×192 slices) using a Siemens 3T scanner for co-registration. Using the combination of each patient's T1-weighted MR image and PET image, every potential necrosis region and tumor progression area were identified within scan. These areas can appear similar at first glance in just the T1-weight image, so each area was referred to as an “abnormality” and was assigned a number to distinguish between them. This collection of all abnormalities within one image is what is classified as one “subject” or “patient.” Next, the surgical pathology data was reviewed in combination with expert clinician analysis to conservatively assign the proper label (TN/0 or TP/1) to all abnormalities within each subject. In cases where there was a combination of TP and TN within one abnormality region, the entire region was then labeled as TP for consistency. There were between one and five abnormalities per subject. After the labeling analysis was concluded, the total number of labeled abnormalities was n=35 areas derived from N=26 scanned subjects. Subsequent processing for each patient was performed with custom tools developed in Matlab (Mathworks Inc., Natick, MA). Image pre-processing including motion correction of dynamic PET data and co-registration into MRI space were per-formed as described4, 5 with bash scripts designed using the FMRIB's Software Library (FSL) tool kit [8-12] (FIG. 13A).

Model corrected blood input function (MCIF). Co-registered volumes were then used in the creation of objective parametric PET maps from a model corrected blood input function (MCIF) corrected for partial volume (PV) averaging and spill-over (SP) contamination as described7, using our techniques developed for rodent [13, 14, 66] and human brains [4, 5, 7].

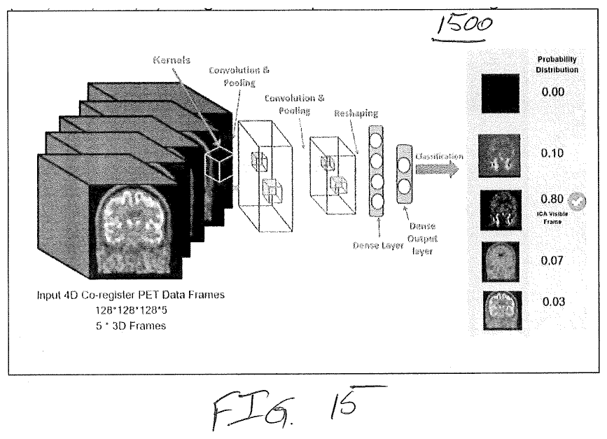

Tumor extraction [7, 15]. 3D masks of abnormality volumes generated from the T1-weighted MR images were semi-automatically segmented using 3D slicer from each subject. In addition to drawing the rough outline of the abnormality area in a few slices, several example slice masks were drawn and entered into the program as “seeds”. Using pre-trained processes, 3D slicer then expanded these seeds automatically to collect the entire abnormality area. Gaussian smoothing was then semi-automatically performed to get both a more conservative and realistic area mask of the abnormality. Masks were then verified by clinical experts. These masks were dropped onto co-registered PET Ki, SUV, and T1-weighted MRI images to mask out the tumor. 3D parametric PET Ki, static PET standardized uptake value (SUV), and MR tumor voxels were extracted in the same image space (FIG. 14). To perform the extraction of 3D tumor voxels while consistently preserving positional information, as a pre-processing step, the images and tumor mask for each subject were co-registered to the SRI 24 atlas [67] with dimensions (240, 240, 155) and re-oriented into Left Posterior Superior (LPS) orientation [68]. This was done by registering the MR to the atlas after performing temporary N4 bias field correction and mutual information for rigid registration using the Cancer Imaging Phenomics Toolkit (CaPTk) [69], then using the computed transformation to bring the other modalities to the same space after which the mask was applied. The masked images were further center cropped to the brain to (170, 170, 120) dimensions by computing a minimum viable bounding box across all datasets to remove unnecessary background voxels and bring focus to the tumors. In FIG. 14, we visualized the extracted tumor voxels for each modality. We observed a higher signal-to-noise ratio showcasing distinct tumor metabolic features within the Ki maps compared to SUVs.

Multimodal architecture. After extracting tumor voxels from different image modalities, we developed a dual-encoder CNN architecture 1300 for multi-modal classification (FIG. 13A). The average dimensions of the tumors are relatively much smaller than the bounding brain region. For these low-dimensional tumor volumes and due to the low number of samples, there is a high probability that employing any SOTA classification network will overfit. Hence, we developed a custom convolutional neural network with a limited number of encoder layers. For each modality, we utilized a shallow 3D convolutional encoder architecture with 3 convolutional layers (kernel size 3, and a number of filters B×2, B×4 and B×4 where the number of base filters B is empirically set to 8). The output latent feature vectors from the encoder of each modality were flattened and fused by concatenation before being fed into the fully connected layers. Additionally, a drop-out layer was added after the first dense layer with a rate of 0.2 for regularization.

Experimental evaluation. Dataset. Our dataset consisted of dynamic 18F-FDG PET scans for 26 subjects as indicated above. T1-weighted MPRAGE MRI scans were also obtained for each subject using the Siemens 3T MR. The scans comprised a total of 35 tumor abnormalities across all subjects along with surgical pathology as the ground truth for each of them as indicated above. By considering multiple disjoint tumor regions present among subjects as distinct inputs, we could increase the number of training samples.

Training setup. Training experiments were performed with both single and multi-modal image input combinations consecutively to perform a comparative evaluation of their classification performance. Adjustments for class imbalance were done by using weighted categorical cross-entropy based on the training distribution of labels (TN:TP balanced class weight ratios 1.95:0.65). For our cross-validation approach, we selected Leave-one-out cross-validation (LOOCV), given the low sample size to perform a less biased and thorough measure of test metrics while utilizing most of the training data. Training is performed with the Adam optimizer and learning rate 10-5 with a batch size of 2, consistently across all iterations for each experiment on a single P100 GPU with 16 GB VRAM. For evaluation, we computed the accuracy, recall, and precision over the entire set of test predictions (35 test samples) across all leave-one-out iterations.

Results Analysis and Discussion. Table 4 showcases classification metrics of our model, compared with the baseline experiments. Our metrics clearly showed that the combination of anatomical differences ingrained in conventional MRI and metabolic differences captured by parametric PET Ki maps yields fairly high classification performance (best accuracy of 0.74) compared to other modalities. Moreover, this also showed that the dual-encoder architecture, which encodes image modalities independently before fusing features out performs compared to the simultaneous dual-channel and single-encoder architectures across all test metrics. Comparing image modalities independently trained with single encoder CNNs, we also observed that Ki alone performs better than MR and SUV with accuracies 0.71, 0.65, and 0.56, respectively. Although we do not see drastic improvements, there is an incremental increase in the accuracy and recall between 4-5%. Single and dual encoder's similar accuracies can, however, be attributed to the lower sample size. Parametric PET Ki which models underlying glucose transport into the tumors is superior to static PET SUV. The computed sensitivity, specificity and AUROC for Ki+MR in the dual-encoder model were 0.84, 0.44 and 0.73 respectively. The lower accuracy, specificity and AUROC are probably due to limited sample size and thereby resulting class imbalance (TP vs TN).

Sex of patients. Current epidemiological data indicate that sex differences exist in patients with GBM and is more prevalent in males than in females [70, 71]. Based on our preliminary studies (35 patients) where we recruited adult patients from either gender (˜66% males and 34% females) [7, 15, 45] and will recruit patients of both genders 18 years and above for this study.

Rationale: Dynamic fluoro-2-deoxy-D-glucose positron emission tomography (dFDG-PET) is an emerging ex-tension of existing static PET imaging technology. dFDG-PET allows for the measurement of time dependent information on glucose uptake in the brain. Quantitative analysis of dFDG-PET scans requires the blood input function (IDIF), which describes the level of radiotracer within the blood that is available for tissues to use, as a means of calibrating regional dynamic glucose changes. While in animal models, the IDIF is captured through arterial sampling, a non-invasive approach is preferred for humans so as to avoid the associated risks (e.g., arterial occlusion and infection) with arterial sampling 72. Manual segmentation of the internal carotid arteries (ICA) is the current gold standard target for deriving the IDIF, which, in turn, is used to calibrate glucose kinetics of the human dFDG-PET. The main goal of this aim is to develop a fully automatic pipeline using deep learning that can facilitate end-to-end selection of the appropriate frame for segmentation of ICA (for derivation of IDIF) and prediction of blood input (with partial volume corrections) for consistent, unbiased and efficient computations of parametric FDG brain PET maps.

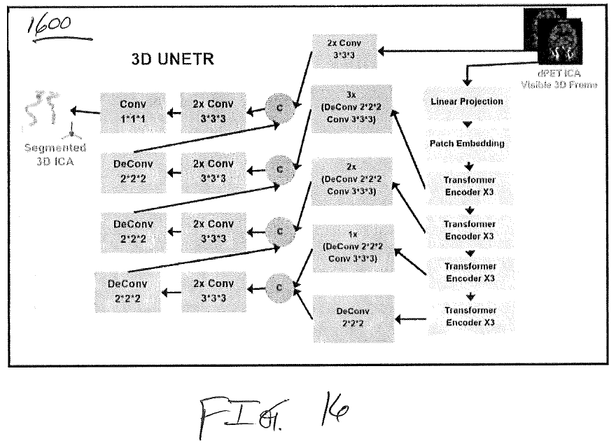

CNN based Frame selection (Frame-net) [18] (FIG. 15). Post motion correction and co-registration, we will focus on segmenting the internal carotid arteries (ICA) within the early frames of the dynamic sequence using a 3D CNN referred to as ‘Frame-net’ to identify the temporal frame displaying the ICA (FIG. 10). This will be an improvement over algorithmic selection of ICA (FIG. 7). For this experiment, five temporal frames selected (FIG. 15 for analysis from each patients' files will be loaded using the Ni Babel library, and the images preprocessed to ensure consistency in dimensions and intensity. Each file will be resized to a uniform shape of (128, 128, 128, 5) using linear interpolation. This resizing will ensure that all images has the same spatial resolution and temporal length. The images will then be normalized using the MinMaxScaler to scale pixel values be-tween 0 and 1. The architecture of Frame-net will include the input layer, convolution layers, dropout layers, dense layers and a final softmax layer to output class probabilities (FIG. 10). Preliminary work18 consisted of 50 brain dynamic PET scans in MR space (4DPETinMR), with each scan having multiple temporal frames for 60-minutes. The Input layer accepted inputs of shape (128, 128, 128, 5) as described above. Here, the five frames were treated as five channels, similar to how RGB channels are handled in 2D CNNs. The convolutional layers had five convolutional layers with increasing filter sizes (64, 128, 128, 256, 256) and kernel sizes of (3, 3, 3), each followed by ReLU activation and max pooling layers (2, 2, 2). Dropout regularization with rates between 0.2 and 0.3 were applied to prevent overfitting. Two dense layers with 512 and 256 neurons, respectively, followed by a final softmax layer output the class probabilities. The model was com-piled using the Adam optimizer and categorical cross-entropy loss with total 4,508,933 params and trainable params: 4,508,933 params. In this preliminary work accuracy was used as the primary metric for evaluation. Training was performed over 100 epochs with a batch size of 2 to handle computational constraints. Each fold's best model, determined by the highest validation accuracy, was saved for further analysis. The 3D CNN model with temporal information tested through a 5-fold cross-validation method reached an average validation accuracy of 86.11%. The model will be retrained on the new data sets collected in this application and rigorously tested through a 10-fold cross-validation to improve validation accuracy.

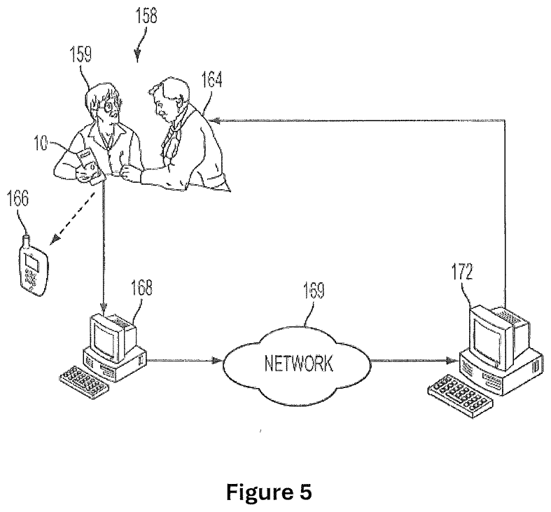

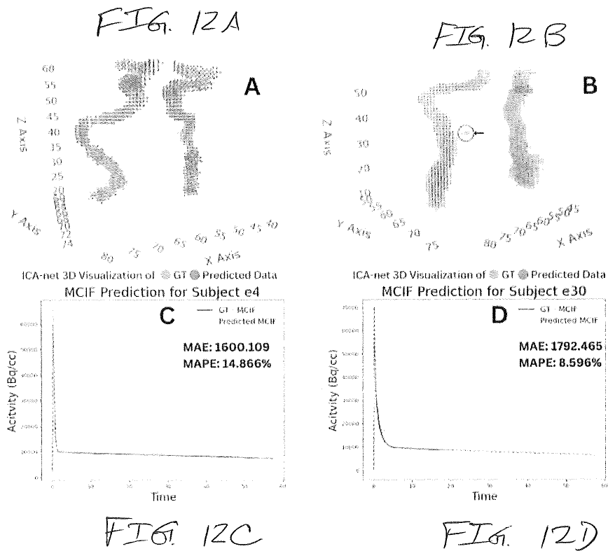

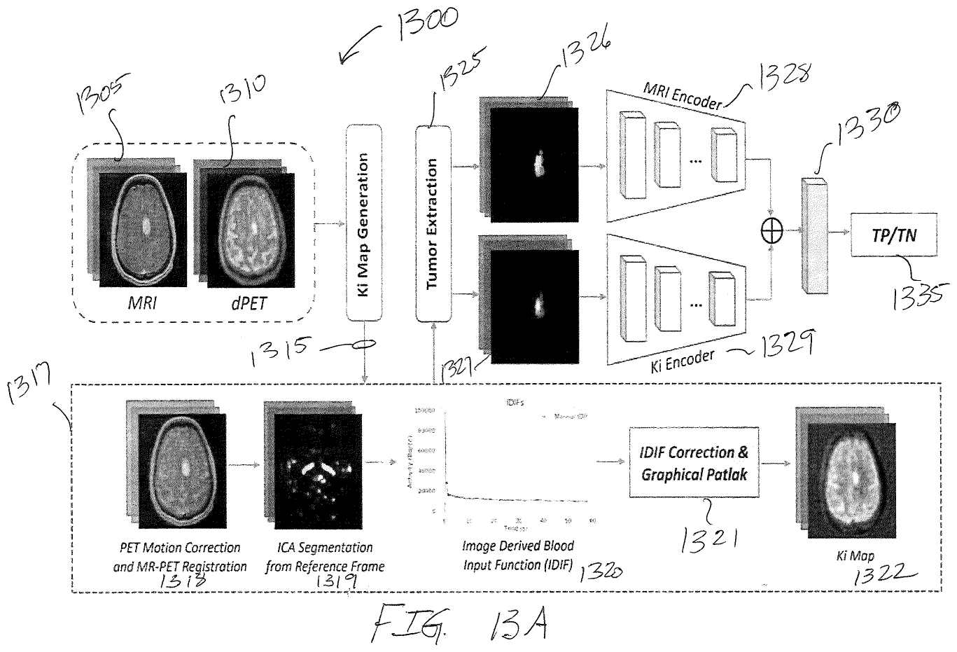

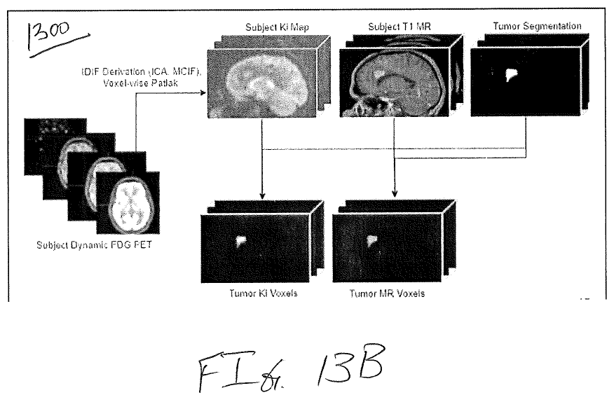

UNETR based ICA segmentation (ICA-net) 18 (FIG. 16). The proposed ICA-net (FIG. 16) is a customized UNETR 20 model designed for the segmentation of ICA for 3D dFDG-PET scans. The selected ICA visible frames by the 3D CNN classifier will be preprocessed by resizing to 128×128×128 voxels and normalizing. Data augmentation will include random rotations, axial flips, and zooms as described in our recent works19. The pre-processing of ICA annotations, derived from a semi-automated segmentation method, will include binarization of the volume data as described in our recent publication (BPEX 2024)19. Both images and labels will undergo the augmentations. Preliminary work employed 10-fold cross-validation for robust model evaluation (FIG. 12). The UNETR model, tailored for 3D segmentation, was trained using the Adam optimizer. Performance was assessed using the Dice coefficient and Jaccard index. Loss Function 73: Our preliminary model employed a combined loss function of Tversky and Cross-Entropy losses to enhance sensitivity and specificity amidst class imbalance. The Tversky Loss, adjusted with alpha and beta at 0.5, and including a softmax layer for multi-class tasks, complements the Cross-Entropy Loss that refines pixel-wise classification. Losses were equally weighted (0.5 each), forming a composite loss. This dual approach ensured balanced learning from both geometry-focused and distribution-focused perspectives, aiming to optimize segmentation accuracy. The ICA-net achieved a notable average Dice score of 83.99% and an Intersection over Union (IoU) of 72.51% across all evaluated scans. The next phase involved computing IDIF, which monitors changes in intensity within the ICA region. To generate the parametric brain PET maps, MCIF was computed by optimizing the IDIF derived from the ICA, to account for partial volume recovery of the blood input5. Preliminary work using the RNN (LSTM plus GRU frameworks) as described earlier (FIG. 5) for mapping IDIF to MCIF employed 10-fold cross validation, compiled with the Adam optimizer and trained on a MSE loss function. MCIF-net underwent fine-tuning over 1000 epochs with a batch size of 32 as described earlier (FIG. 5). A combination of Bi-directional GRU and LSTM yielded the best results with an MAE of 0.1014 and an MSE of 0.1307. The models will be retrained with new data sets collected in this application for efficient parametric brain PET mapping.

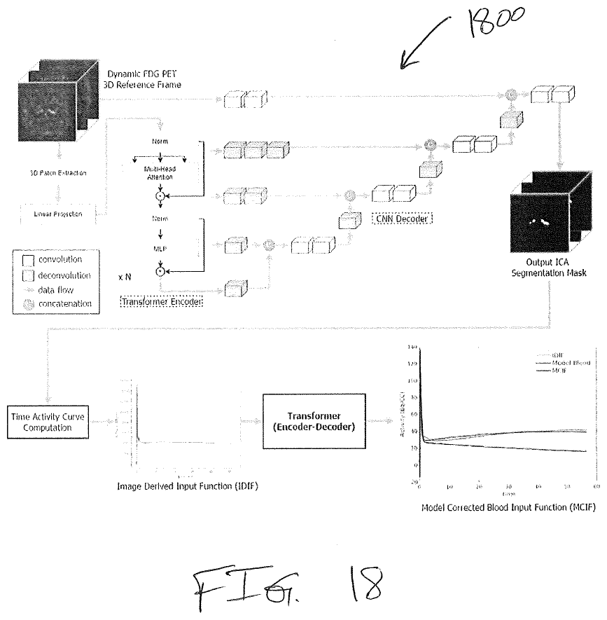

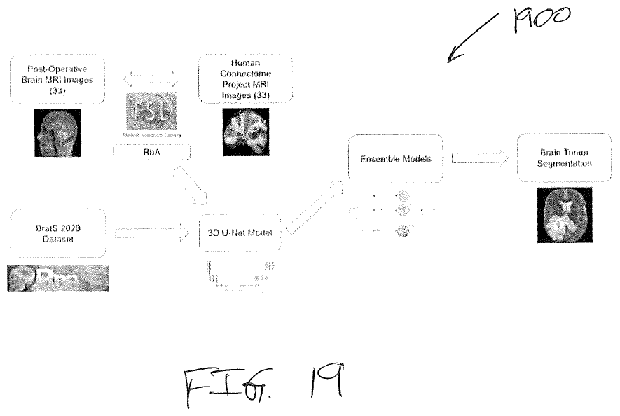

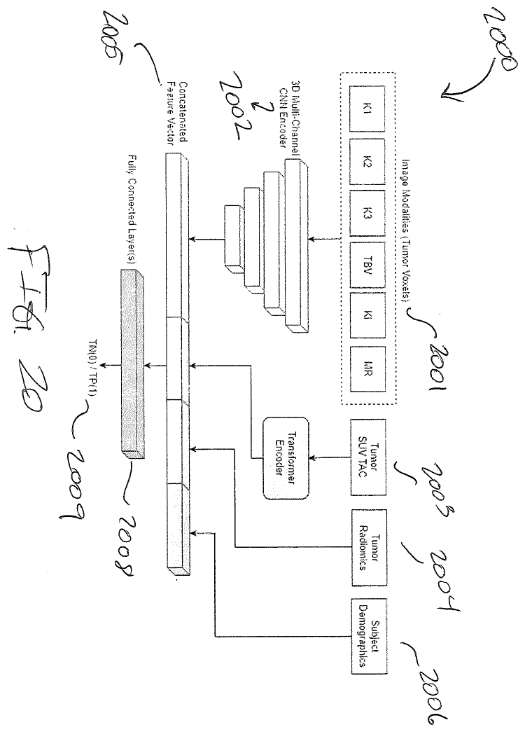

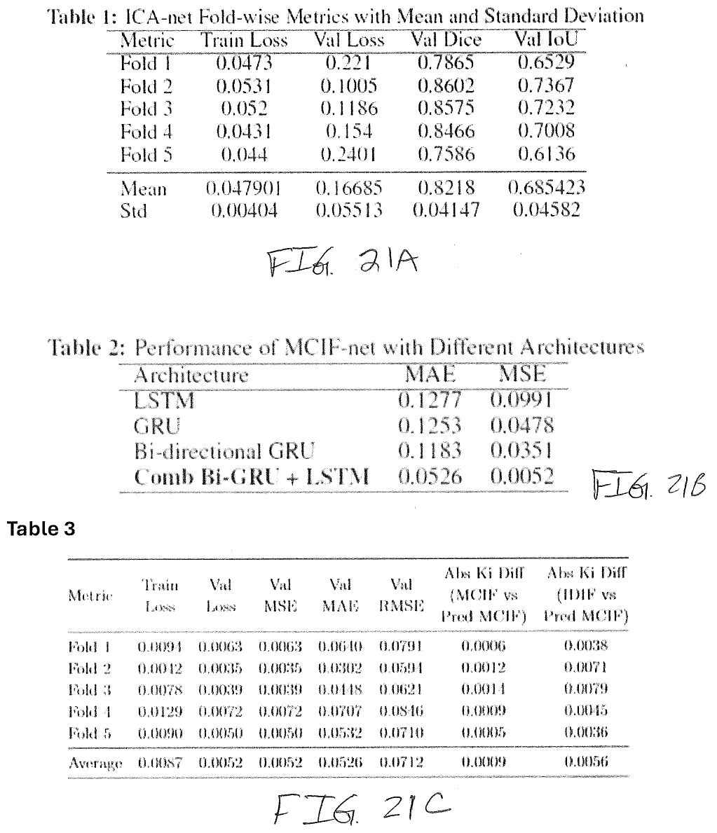

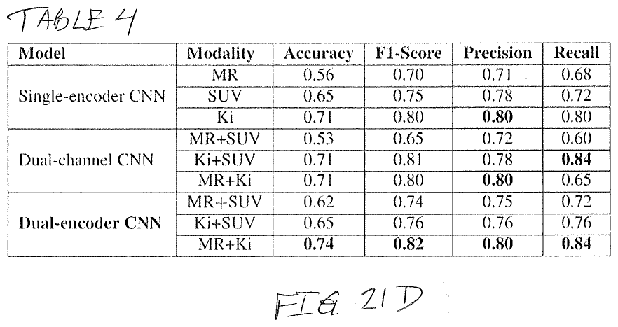

ICA segmentation and blood input prediction with partial volume corrections (FIG. 18). A UNETR 20 based model (hybrid 3D UNet based convolutional neural net-work with transformer layers), as visualized in FIGS. 11 and 13 will be further developed and re-trained to learn the structure of the ICA for auto-mated segmentation Given the 3D reference frame as input (Frame-net; FIG. 16) 18, this UNETR, once trained, will output segmentation mask for ICA. Data augmentation and 10-fold cross-validation approaches as described above will be used to handle low training sample size imaging data. For evaluation, segmentation metrics such as Dice Similarity Co-efficient (DSC), Intersection over Union (IoU) score, and Hausdorff Distance (HD) would be utilized as described above. This will be followed by a time series computation step, where the volume of interest (VOI) segmentation mask is convolved over the raw dynamic PET series to compute the IDIF from the ICA segmentation. For a more downstream comparison of the generated segmentation, the generated TACs from the predictions will be compared for similarity with the ground truth segmentation TACs using the root mean-squared error (RMSE), mean absolute percentage error (MAPE) and area under curve (AUC) as described19, 54, 55. To correct the generated IDIF for the effects of spillover contamination and partial volume averaging, the IDIF will be utilized in a multi-parameter optimization model developed for dynamic FDG PET for model MCIF generation5, 14, 74. Similarly, we will utilize the IDIF TAC generated from the predicted segmentations as input features for another supervised deep learning model that learns how to perform this input function correction. Given the IDIF as input and its corresponding MCIF as ground truth for the same human dataset, another time distributed or sequence-sequence trans-former-based network ar-chitecture21 for time-series regression using positional encoding will be formulated to train and predict MCIF for test data and compared to reference modeled MCIF. For evaluation of the similarity of the predicted MCIFs against the model MCIFs, along with time series similarity metrics mentioned before to compare the raw TACs, another downstream metric will be computed measuring the absolute difference between model and predicted downstream whole brain parametric Ki maps as described 18, 19, 54. The predicted MCIF will be compared to the model based auto-mated blood input derivation (validated with arterial blood samples) utilizing local means analysis (LMA) as de-scribed46. Downstream prediction of tumor recurrence could be indirect measures of ground truth MCIF.

FIG. 13: Proposed cascade network architectures (UNETR/Transformer). A 3D UNETR based model will be trained to learn the structure and localization of the ICA for automated segmentation. Another sequence-sequence transformer-based network architecture for time-series regression using positional encoding will be formulated to train and predict ground truth MCIF and downstream parametric Ki maps for test data using IDIF as input.

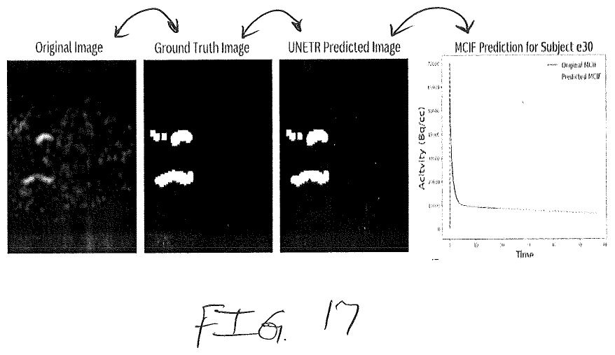

Expected outcomes and alternate strategies: This research expects to develop and train UNETR/Transformer networks to successfully predict MCIF by mapping IDIF to reference model MCIF. With transformer based architecture the plan is to utilize the model with transfer learning to other tracers as well for human dynamic brain PET. We expect to perform better than the state of the art LMA method validated with arterial blood samples for human dynamic FDG brain PET. Alternatively, if the proposed transformer based architecture is unable to learn the underlying representation across datasets for segmentation and lacks performance, we will revert back to the UNETR and RNN models combining LSTM and GRU frameworks as described above (FIGS. 17 and 11).

Rationale: Distinguishing recurrent neoplasm (tumor progression (TP)) from treatment effect (tumor necrosis (TN)) is critical for clinical management decisions in human glioblastoma (GBM). Advanced MRI techniques including diffusion and perfusion imaging have yielded inconsistent and unreliable results for differentiating be-tween these entities. SUV analysis does not reliably differentiate tumor from post-therapy changes in the standard static PET imaging protocol. Preliminary work utilizing dynamic PET with manually segmented brain tumors

and standard classification algorithms (logistic regression and support vector machine) on average PET features and convolution neural network (CNN) on 3D tumor volumes in image space (PET Ki and MR tumor voxels) respectively yielded similar test prediction accuracy of 0.74.45 The lower prediction accuracy for the deep learning model is due to lower sample size and class imbalance between TP and TN. Larger data sets for better vali-dation that represents the entire GBM distribution better to develop a robust multimodal deep learning model that the clinicians can use is therefore needed.

Research Design: This will be a prospective study of 100 high-grade glioma (HGG) or GBM patients aged 18 and older who have undergone chemoradiation therapy with fractionated radiotherapy and temozolomide (for the HGG subjects) and have subsequently developed a new or enlarging enhancing brain lesion or signal abnormality on routine clinical MRI surveillance that raises the differential diagnosis of tumor recurrence versus treatment effect. Both men and women 18 and above will be recruited into this study by Dr. Schiff (Co-I). 60 minute dynamic FDG PET with the TOF Siemens PET imager will be acquired for these patients. Deep learning models described below for brain tumor segmentation and classification will be compared to the criterion standard for determining tumor recurrence versus treatment effect, which will be based on either surgical pathology or an integrated clinical/MRI determination at 3-6 month interval. The prognostic value of classification using multimodal deep learning will also be ascertained by comparing to patient overall survival times.

i) Dynamic FDG PET: Dynamic PET scan will be initiated followed by administration of ˜10 mCi of 18F-FDG and imaged for 60 minutes. Two late venous blood samples will be collected at the end of the 60 minute scan for validation of the image-derived blood input function obtained from the ICA in the brain. The traditional static PET findings (by integrating the last 15 minutes of the dynamic scan) will be evaluated by Dr. Muttikkal (Co-I).

ii) MRI: This will include volumetric T1-weighted, axial T2-weighted, fluid attenuation recovery (FLAIR), gradient-recalled-echo (GRE), and dynamic susceptibility perfusion (DSC-PWI), axial and coronal diffusion-weighted im-aging (DWI), and 3D T1-weighted pre- and post-contrast MR imaging in collaboration with Dr. Patel (Co-I).

iii) Radiomics: Radiomics features will be extracted from the tumor voxels including first-order statistics, 2D and 3D shape-descriptors and texture level features from digital phantom texture matrices in compliance with feature definitions from the Imaging Biomarker Standardization Initiative (IBSI)75-77 using the PyRadiomics27 feature ex-tractor. A preliminary classification experiment was performed using an SVM on 65 radiomics features (obtained from 107 total features with variance thresholding by selecting features with less than or equal to 0 variance) with 70:30% train: test splits over 100 iterations of repeated random subsampling cross validation. The average test accuracies for MR, Ki and SUV were 0.5±0.165, 0.603±0.151 and 0.497±0.167 (min-max scaling) respectively, showcasing the predictive power of these features as well as Ki yielding higher accuracy with a lower error against the other modalities. Another experiment combined all modalities (Ki, MR and SUV) to determine the best leave one out cross validation (LOOCV) test accuracy. Preliminary radiomics analysis revealed 35 features with the best LOOCV test accuracy of 0.74 using SVM (poly) compared to all 321 features (107/modality) (test accuracy=0.44) combining PET and MR modalities.

iv) Surgical pathology: Report will be obtained from Drs. Schiff and Sheehan (Co-I) in Neurology and Neuro-surgery and considered the gold standard for determining presence of recurrent tumor or radiation necrosis.

Proposed models for brain tumor segmentation and improved classification. Brain tumor seg-mentation: A 3D U-Net trained on datasets from the Brain Tumor Segmentation (BraTS) challenge23, 24 (the best architecture from the challenge) with is a set of pre-operative MRI, specifically for the whole tumor label combined, and an ensemble of the best models will be transfer learnt 22 to our post-operative tumor datasets. For this supervised transfer learning task, either a divergence-based domain adaptation 78 will be performed or alternatively, we will revert to fine-tuning with existing weights and new layers for the encoder and decoder heads.

Multimodal brain tumor classification: Multimodal image features focusing on the tumor using masks from the prior method will be generated for all subjects similarly, including parametric PET map voxels specifically those for Ki, individual rate constants K1-K3 and total blood volume (TBV), along with SUV and MR voxels. Similarly, other features such as the tumor tissue time-activity curve (TAC) will be obtained by convolving the tumor segmentation mask over the dynamic PET image and standardized against weight and injected dose. The resultant image modalities, tumor SUV TAC along with tumor radiomics and demographics for the tumor source subject will be fed into a multimodal network for binary brain tumor classification (MMDL) as shown in FIG. 15. Each feature type has to be encoded differently. For the image feature encoder module, we will experiment with both a CNN-based multi-channel encoder and a series of separate CNN encoders alternatively. To encode the time-series tu-mor SUV TACs, a Transformer based encoder will be used. Low-dimensional latent space features extracted from all feature encoders and other low-dimensional inputs will be fused together by concatenation and fed into a series of fully connected layers for the final classification. The proposed deep learning architectures proposed in aims 1, transfer learning for brain tumor segmentation and multimodal learn-ing will be developed in collaboration.

Proposed multimodal deep learning (MMDL) architecture for classification between tumor progression (TP) and treated related necrosis (TN). The MMDL features include parametric PET, MR, radiomics and subject demographics.

### REFERENCE LIST

(1) American Association of Neurological Surgeons 2016; Available at: URL: http://www.aans.org/patient%20information/conditions%20and%20treatments/glioblastoma%20multiforme.aspx.
(2) Stupp R, Mason W P, van den Bent M J, Weller M, Fisher B, Taphoorn M J, Belanger K, Brandes A A, Marosi C, Bogdahn U, Curschmann J, Janzer R C, Ludwin S K, Gorlia T, Allgeier A, Lacombe D, Cairn-cross J G, Eisenhauer E, Mirimanoff R O. Radiotherapy plus concomitant and adjuvant temozolomide for glioblastoma. N Engl J Med 2005 Mar. 10; 352(10):987-96.
(3) Kim Y H, Oh S W, Lim Y J, Park C K, Lee S H, Kang K W, Jung H W, Chang K H. Differentiating radiation necrosis from tumor recurrence in high-grade gliomas: assessing the efficacy of 18F-FDG PET, 11C-methionine PET and perfusion MRI. Clin Neurol Neurosurg 2010 November; 112(9):758-65.
(4) Quigg M, Kundu B K. Dynamic FDG-PET demonstration of functional brain abnormalities. Annals of Clinical and Translational Neurology 2022; 9(9):1487-97.
(5) Seshadri V, Zarroli K A, Schetlick R S, Massey J C, Reyes J M, Eluvathingal Muttikal T J, Patrie J T, Berr S S, Fountain N B, Kundu B K, Quigg M. Dynamic FDG-PET in localization of focal epilepsy: A pilot study. Epilepsy Behav 2021 September; 122:108204.
(6) Conti M, Bendriem B. The new opportunities for high time resolution clinical TOF PET. Clinical and Translational Imaging 2019; 7:139-47.
(7) Schetlick R S, Muttikkal T E, Jose R E, Batchala P, Doanhue J, Patel S, Schiff D, Kundu B K. Paramet-ric FDG PET Quantification, Segmentation and Classification of Primary Brain Tumors in Human GBM. 2021 IEEE Nuclear Science Symposium and Medical Imaging Conference (NSS/MIC), 2021, pp. 1-5, doi: 10.1109/NSS/MIC44867.2021.9875767.
(8) Jenkinson M, Smith S. A global optimisation method for robust affine registration of brain images. Med Image Anal 2001; 5:143-56.
(9) Jenkinson M, Beckmann C F, Behrens T E, Woolrich M W, Smith S M. FSL. Neuroimage 2012 Aug. 15; 62(2):782-90.
(10) Jenkinson M, Bannister P, Brady M, Smith S. Improved optimization for the robust and accurate linear registration and motion correction of brain images. Neuroimage 2002; 17:825-41.
(11) Smith S M, Jenkinson M, Woolrich M W, Beckmann C F, Behrens T E, Johansen-Berg H, Bannister P R, De L M, Drobnjak I, Flitney D E, Niazy R K, Saunders J, Vickers J, Zhang Y, De S N, Brady J M, Matthews P M. Advances in functional and structural MR image analysis and implementation as FSL. Neuroimage 2004; 23 Suppl 1: S208-S219.
(12) Woolrich M W, Jbabdi S, Patenaude B, Chappell M, Makni S, Behrens T, Beckmann C, Jenkinson M, Smith S M. Bayesian analysis of neuroimaging data in FSL. Neuroimage 2009 March; 45(1 Suppl):S173-S186.
(13) Massey J C, Seshadri V, Paul S, Minczuk K, Molinos C, Li J and Kundu B K. Model Corrected Blood Input Function to Compute Cerebral FDG Uptake Rates from Dynamic Total-Body PET Images of Rats in vivo. Front. Med. 2021; 8:618645.
(14) Zhong M, Kundu B K. Optimization of a Model Corrected Blood Input Function From Dynamic FDG-PET Images of Small Animal Heart In Vivo. Ieee Transactions on Nuclear Science 2013 Oct. 9; 60(5):3417-22.
(15) Hossain T, Qureshi Z, Jayakumar N, Muttikkal T E, Patel S, Schiff D, Quigg M, Zhang M and Kundu B K, Multimodal Deep Learning to Differentiate Tumor Recurrence from Treatment Effect in Human Gli-oblastoma,” 2023 IEEE 20th International Symposium on Biomedical Imaging (ISBI), Cartagena, Co-lombia, 2023, pp. 1-4, doi: 10.1109/ISBI53787.2023.10230599. arxiv.org/abs/2302.14124.
(16) Kundu B K, William Terrell, Zoraiz Qureshi, et al. Dynamic FDG PET for multimodal classification of tumor progression from treatment effect in human glioblastoma: Regression, Radiomics and Deep learning. J Nucl Med June 2024, 65 (supplement 2) 241157 jnm.snmjournals.org/content/65/supple-ment_2/241157
(17) Lei F, Liu X, Dai Q, et al. Shallow convolutional neural network for image classification. S N Appl Sci 2020; 2(97).
(18) Chavan R, Terrell W, Berr S, Muttikkal T, Quigg M, Kundu B K. AI framework with 3D CNN-assisted UNETR and RNN for blood input compute with partial volume corrections for automated parametric FDG brain PET mapping. Oral Presentation, World Molecular Imaging Congress (Sep. 9-13, 2024. Montreal, Canada). Scientific Session: Computational & Data Science. Special Session: Intelligent Imaging: Harnessing AI for Enhanced Detection and Diagnosis. Affiliation: University of Vir-ginia www.xcdsystem.com/wmis/program/41mRX3V/in-dex.cfm?pgid=2968&sid=28467&abid=110869
(19) Chavan R, Hyman G, Qureshi Z, Jayakumar N, Terrell W, Wardius M, Berr S, Schiff D, Fountain N, Eluvathingal M T, Quigg M, Zhang M, Kundu B K. An end-to-end deep learning pipeline to derive blood input with partial volume corrections for automated parametric brain PET mapping. Biomed Phys Eng Express 2024 Aug. 19; 10(5):055028. doi: 10.1088/2057-1976/ad6a64.
(20) Hatamizadeh A, Tang Y, Nath V, Yang D, Myronenko A, Landman B, Roth HR, Xu D. UNETR: Trans-formers for 3D Medical Image Segmentation. arXiv:2103.10504v3 [eess.IV] 9 Oct. 2021.
(21) Ahmed S, Nielsen I E, Tripathi A, Siddiqui H, Rasool G, Ramachandran R P. Transformers in Time-se-ries Analysis: A Tutorial. arXiv:2205.01138v1 [cs.LG] 28 Apr. 2022.
(22) Ghaffari M, Samarasinghe G, Jameson M, Aly F, Holloway L, Chlap P, Koh E S, Sowmya A, Oliver R. Automated post-operative brain tumour segmentation: A deep learning model based on transfer learn-ing from pre-operative images. Magn Reson Imaging 2022 February; 86:28-36.
(23) BraTS. braintumorsegmentation.org/
(24) Kofler F, Berger C, Waldmannstetter D, Lipkova J, Ezhov I, Tetteh G, Kirschke J, Zimmer C, Wiestler B, Menze B H. BraTS Toolkit: Translating BraTS Brain Tumor Segmentation Algorithms Into Clinical and Scientific Practice. Front Neurosci 2020; 14:125.
(25) Dai Y, Gao Y, Liu F. TransMed: Transformers Advance Multi-Modal Medical Image Classification. Diag-nostics (Basel) 2021; 11(8):1384.
(26) Kotrotsou A, Zinn P O, Colen R R. Radiomics in Brain Tumors: An Emerging Technique for Characteri-zation of Tumor Environment. Magn Reson Imaging Clin N Am 2016 November; 24(4):719-29.
(27) van Griethuysen J J M, Fedorov A, Parmar C, Hosny A, Aucoin N, Narayan V, Beets-Tan R G H, Fillion-Robin J C, Pieper S, Aerts H J W L. Computational Radiomics System to Decode the Radiographic Phe-notype. Cancer Res 2017 Nov. 1; 77(21):e104-e107.
(28) Yang I, Huh N G, Smith Z A, Han S J, Parsa A T. Distinguishing glioma recurrence from treatment effect after radiochemotherapy and immunotherapy. Neurosurg Clin N Am 2010 January; 21(1):181-6.
(29) Barajas R F, Jr., Chang J S, Segal M R, Parsa A T, McDermott M W, Berger M S, Cha S. Differentiation of recurrent glioblastoma multiforme from radiation necrosis after external beam radiation therapy with dynamic susceptibility-weighted contrast-enhanced perfusion MR imaging. Radiology 2009 Novem-ber; 253(2):486-96.
(30) Bobek-Billewicz B, Stasik-Pres G, Majchrzak H, Zarudzki L. Differentiation between brain tumor recur-rence and radiation injury using perfusion, diffusion-weighted imaging and MR spectroscopy. Folia Neu-ropathol 2010; 48(2):81-92.
(31) Hu L S, Baxter L C, Smith K A, Feuerstein B G, Karis J P, Eschbacher J M, Coons S W, Nakaji P, Yeh R F, Debbins J, Heiserman J E. Relative cerebral blood volume values to differentiate high-grade glioma re-currence from posttreatment radiation effect: direct correlation between image-guided tissue histo-pathology and localized dynamic susceptibility-weighted contrast-enhanced perfusion MR imaging measurements. AJNR Am J Neuroradiol 2009 March; 30(3):552-8.
(32) Wang S, Martinez-Lage M, Sakai Y, Chawla S, Kim S G, onso-Basanta M, Lustig R A, Brem S, Mohan S, Wolf R L, Desai A, Poptani H. Differentiating Tumor Progression from Pseudoprogression in Patients with Glioblastomas Using Diffusion Tensor Imaging and Dynamic Susceptibility Contrast MRI. AJNR Am J Neuroradiol 2016 January; 37(1):28-36.
(33) Hein P A, Eskey C J, Dunn J F, Hug E B. Diffusion-weighted imaging in the follow-up of treated high-grade gliomas: tumor recurrence versus radiation injury. AJNR Am J Neuroradiol 2004 Febru-ary; 25(2):201-9.
(34) Chan Y L, Yeung D K, Leung S F, Chan P N. Diffusion-weighted magnetic resonance imaging in radia-tion-induced cerebral necrosis. Apparent diffusion coefficient in lesion components. J Comput Assist Tomogr 2003 September; 27(5):674-80.
(35) Sugahara T, Korogi Y, Tomiguchi S, Shigematsu Y, Ikushima I, Kira T, Liang L, Ushio Y, Takahashi M. Posttherapeutic intraaxial brain tumor: the value of perfusion-sensitive contrast-enhanced MR imaging for differentiating tumor recurrence from nonneoplastic contrast-enhancing tissue. AJNR Am J Neuro-radiol 2000 May; 21(5):901-9.
(36) Sundgren P C, Fan X, Weybright P, Welsh R C, Carlos R C, Petrou M, Mckeever P E, Chenevert T L. Dif-ferentiation of recurrent brain tumor versus radiation injury using diffusion tensor imaging in patients with new contrast-enhancing lesions. Magn Reson Imaging 2006 November; 24(9):1131-42.
(37) Kosaka N, Tsuchida T, Uematsu H, Kimura H, Okazawa H, Itoh H. 18F-FDG PET of common enhanc-ing malignant brain tumors. AJR Am J Roentgenol 2008 June; 190(6):W365-W369.
(38) Boellaard R. Standards for PET image acquisition and quantitative data analysis. J Nucl Med 2009 May; 50 Suppl 1:11S-20S.
(39) Britz-Cunningham S H, Millstine J W, Gerbaudo V H. Improved discrimination of benign and malignant lesions on FDG PET/CT, using comparative activity ratios to brain, basal ganglia, or cerebellum. Clin Nucl Med 2008 October; 33(10):681-7.
(40) Nozawa A, Rivandi A H, Kesari S, Hoh C K. Glucose corrected standardized uptake value (SUVgluc) in the evaluation of brain lesions with 18F-FDG PET. Eur J Nucl Med Mol Imaging 2013 Apr. 10.
(41) Chen W, Silverman D H, Delaloye S, Czernin J, Kamdar N, Pope W, Satyamurthy N, Schiepers C, Cloughesy T. 18F-FDOPA PET imaging of brain tumors: comparison study with 18F-FDG PET and evaluation of diagnostic accuracy. J Nucl Med 2006 June; 47(6):904-11.
(42) Wardak M, Schiepers C, Cloughesy T F, Dahlbom M, Phelps M E, Huang S C. (1)(8)F-FLT and (1)(8)F-FDOPA PET kinetics in recurrent brain tumors. Eur J Nucl Med Mol Imaging 2014 June; 41(6):1199-209.
(43) Dunet V, Pomoni A, Hottinger A, Nicod-Lalonde M, Prior J O. Performance of 18F-FET versus 18F-FDG-PET for the diagnosis and grading of brain tumors: systematic review and meta-analysis. Neuro Oncol 2016 March; 18(3):426-34.
(44) Juhasz C, Dwivedi S, Kamson D O, Michelhaugh S K, Mittal S. Comparison of Amino Acid Positron Emission Tomographic Radiotracers for Molecular Imaging of Primary and Metastatic Brain Tumors. Mol Imaging 2014 Aug. 1; 13(6):7290201400015.
(45) Kundu B K, William Terrell, Rugved Chavan, et al. Interictal dynamic FDG PET in focal epilepsy. J Nucl Med. 2024 65 (supplement 2) 241167. jnm.snmjournals.org/content/65/supplement_2/241167
(46) Zanotti-Fregonara P, Maroy R, Comtat C, Jan S, Gaura V, Bar-Hen A, Ribeiro M J, Trebossen R. Com-parison of 3 methods of automated internal carotid segmentation in human brain PET studies: applica-tion to the estimation of arterial input function. J Nucl Med 2009 March; 50(3):461-7.
(47) Qureshi Z, Peng P, Zhang M, Kundu B K. Machine learning derived model corrected blood input func-tion from dynamic FDG PET images of rat heart. J Nucl Med Abstract. 2022; 63 (supp 2) 3229. jnm.snmjournals.org/content/63/supplement_2/3229
(48) Kuttner S, Wickstrom K K, Kalda G, Dorraji S E, Martin-Armas M, Oteiza A, Jenssen R, Fenton K, Sundset R, Axelsson J. Machine learning derived input-function in a dynamic (18)F-FDG PET study of mice. Biomed Phys Eng Express 2020 Jan. 13; 6(1):015020.
(49) Traub-Weidinger T, Muzik O, Sundar L K S, ull-Watschinger S, Beyer T, Hacker M, Hahn A, Kasprian G, Klebermass E M, Lanzenberger R, Mitterhauser M, Pilz M, Rausch I, Rischka L, Wadsak W, Pataraia E. Utility of Absolute Quantification in Non-lesional Extratemporal Lobe Epilepsy Using FDG PET/MR Im-aging. Front Neurol 2020; 11:54.
(50) Shah V. Automatic landmarking and parsing of human anatomy (ALPHA) for innovative and smart MI applications. www siemens-healthineers com/en-us/molecular-imaging/mi-clinical-corner/whitepa-pers/automatic-landmarking-and-parsing-human-anatomy 2021.
(51) Moradi H, Vashistha R, Ghosh S, O'Brien K, Hammond A, Rominger A, Sari H, Shi K, Vegh V, Reutens D. Automated extraction of the arterial input function from brain images for parametric PET studies. EJNMMI Res 2024 Apr. 1; 14(1):33.
(52) Gao Y, Xiao X, Han B, Li G, Ning X, Wang D, Cai W, Kikinis R, Berkovsky S, Di I A, Zhang L, Ji N, Liu S. Deep Learning Methodology for Differentiating Glioma Recurrence From Radiation Necrosis Using Multimodal Magnetic Resonance Imaging: Algorithm Development and Validation. J MIR Med Inform 2020 Nov. 17; 8(11):e19805.
(53) Park Y W, Choi D, Park J E, Ahn S S, Kim H, Chang J H, Kim S H, Kim H S, Lee S K. Differentiation of re-current glioblastoma from radiation necrosis using diffusion radiomics with machine learning model de-velopment and external validation. Sci Rep 2021 Feb. 3; 11(1):2913.
(54) Jayakumar N, Qureshi Z, Schiff D, Quigg M, Zhang M, Kundu B K. A deep learning pipeline for para-metric FDG brain PET mapping in localization of focal epilepsy. Journal of Nuclear Medicine June 2023, 64 (supplement 1) P808. jnm.snmjournals.org/content/64/supplement_1/P808
(55) Qureshi Z, Hyman G, Schiff D, Quigg M, Zhang M, Kundu B K. 3D U-Net based internal carotid artery segmentation for derivation of image derived blood input function for human brain dynamic FDG PET. World Molecular Imaging Congress (Oral presentation). Oct. 1, 2022. www.xcdsys-tem.com/wmis/program/cJi6upE/index.cfm?pgid=2765&sid=25510&abid=93680
(56) Hochreiter S, Schmidhuber J. Long short-term memory. Neural Comput 1997 November 15; 9(8):1735-80.
(57) Schmidhuber J, Gers F, Eck D. Learning nonregular languages: a comparison of simple recurrent net-works and LSTM. Neural Comput 2002 September; 14(9):2039-41.
(58) Ye A. Long Short-Term Memory Networks Are Dying: What's Replacing It? 2020. medium.com/mlearn-ing-ai/long-short-term-memory-networks-are-dying-whats-replacing-it-5ff3a99399fe
(59) Bijoy Kundu, et al; Automation of the blood input function computation pipeline for dynamic fdg pet for human brain using machine learning. WO2023196474A1. WIPO (PCT). Application PCT/US2023/017697 patents.google.com/patent/WO2023196474A1
(60) Neumann K D, Seshadri V, Thompson X D, Broshek D K, Druzgal J, Massey J C, Newman B, Reyes J, Simpson S R, McCauley K S, Patrie J, Stone J R, Kundu B K, Resch J E. Microglial activation persists beyond clinical recovery following sport concussion in collegiate athletes. Front Neurol 2023; 14:1127708.
(61) Neumann K D, Broshek D K, Newman B T, Druzgal T J, Kundu B K, Resch J E. Concussion: Beyond the Cascade. Cells 2023 Aug. 22; 12(17).
(62) Schetlick R S, Muttikkal T E, Jose R E, Batchala P, Doanhue J, Patel S, Schiff D, Kundu B K. Paramet-ric FDG PET Quantification, Segmentation and Classification of Primary Brain Tumors in Human GBM. 2021 IEEE Nuclear Science Symposium and Medical Imaging Conference (NSS/MIC), 2021, pp. 1-5, doi: 10.1109/NSS/MIC44867.2021.9875767.
(63) Özgün Çiçek, Ahmed Abdulkadir, S S L, Thomas Brox, Olaf Ronneberger. 3d u-net: Learning dense vol-umetric segmentation from sparse annotation. 2016.
(64) Sandfort V, Yan K, Pickhardt P J, Summers R M. Data augmentation using generative adversarial net-works (CycleGAN) to improve generalizability in CT segmentation tasks. Sci Rep 2019 Nov. 15; 9(1):16884.
(65) Kyunghyun Cho, Bart van Merrienboer., Caglar Gulcehre, Dzmitry Bahdanau, Fethi Bougares, Holger Schwenk, Yoshua Bengio. On the properties of neural machine translation: Encoder-decoder approaches. 2014 p. 103-11.
(66) Kundu B K, Antkowiack P, Huang T et al. Dynamic FDG PET imaging of the brain in a mouse model of dystonia. J Nucl Med abstract 2015; 56[3], 1554.
(67) Rohlfing T, Zahr N M, Sullivan E V, Pfefferbaum A. The SRI24 Multi-Channel Brain Atlas: Construction and Applications. Proc SPIE Int Soc Opt Eng 2008; 6914:691409.
(68) Gordon L. Kindlmann. An self-contained explanation of image orientation and the “measurement frame”, with connections to the NRRD format (Version 0.5). University of Chicago. 2010.
(69) Davatzikos C, Rathore S, Bakas S, Pati S, Bergman M, Kalarot R, Sridharan P, Gastounioti A, Jahani N, Cohen E, Akbari H, Tunc B, Doshi J, Parker D, Hsieh M, Sotiras A, Li H, Ou Y, Doot R K, Bilello M, Fan Y, Shinohara R T, Yushkevich P, Verma R, Kontos D. Cancer imaging phenomics toolkit: quantita-tive imaging analytics for precision diagnostics and predictive modeling of clinical outcome. J Med Im-aging (Bellingham) 2018 January; 5(1):011018.
(70) Ostrom Q T, Price M, Neff C, Cioffi G, Waite K A, Kruchko C, Barnholtz-Sloan J S. CBTRUS Statistical Report: Primary Brain and Other Central Nervous System Tumors Diagnosed in the United States in 2016-2020. Neuro Oncol 2023 Oct. 4; 25(12 Suppl 2):iv1-iv99.
(71) Sun T, Plutynski A, Ward S, Rubin J B. An integrative view on sex differences in brain tumors. Cell Mol Life Sci 2015 September; 72(17):3323-42.
(72) Oertel B G, Vermehren J, Zimmermann M, Huynh T T, Doehring A, Ferreiros N, Senzel S, Schmitz-Rixen T, Erbe M, Geisslinger G, Harder S, Angst M S, Lotsch J. Necessity and risks of arterial blood sampling in healthy volunteer studies. Clin Pharmacokinet 2012 Oct. 1; 51(10):629-38.
(73) Jadon S. A survey of loss functions for semantic segmentation. 2020.
(74) Massey J C, Seshadri V, Paul S, Minczuk K, Molinos C, Li J, Kundu B K. Model Corrected Blood Input Function to Compute Cerebral FDG Uptake Rates From Dynamic Total-Body PET Images of Rats in vivo. Front Med (Lausanne) 2021; 8:618645.
(75) Zwanenburg A. Radiomics in nuclear medicine: robustness, reproducibility, standardization, and how to avoid data analysis traps and replication crisis. Eur J Nucl Med Mol Imaging 2019 Decem-ber; 46(13):2638-55.
(76) Zwanenburg A, Vallieres M, Abdalah M A, Aerts H J W L, Andrearczyk V, Apte A, Ashrafinia S, Bakas S, Beukinga R J, Boellaard R, Bogowicz M, Boldrini L, Buvat I, Cook G J R, Davatzikos C, Depeursinge A, Desseroit M C, Dinapoli N, Dinh C V, Echegaray S, El N, I, Fedorov A Y, Gatta R, Gillies R J, Goh V, Gotz M, Guckenberger M, Ha S M, Hatt M, Isensee F, Lambin P, Leger S, Leijenaar R T H, Lenkowicz J, Lip-pert F, Losnegard A, Maier-Hein K H, Morin O, Muller H, Napel S, Nioche C, Orlhac F, Pati S, Pfaehler E A G, Rahmim A, Rao A U K, Scherer J, Siddique M M, Sijtsema N M, Socarras F J, Spezi E, Steen-bakkers R J H M, Tanadini-Lang S, Thorwarth D, Troost E G C, Upadhaya T, Valentini V, van Dijk L V, van G J, van Velden F H P, Whybra P, Richter C, Lock S. The Image Biomarker Standardization Initiative: Standardized Quantitative Radiomics for High-Throughput Image-based Phenotyping. Radiology 2020 May; 295(2):328-38.
(77) Zwanenburg A, Leger S, Vallières M, Löck S. Image biomarker standardisation initiative-feature defini-tions. 2019; arXiv:1612.07003v11 [cs.CV] 17 Dec. 2019.
(78) Lutao Dai, Tengfei Li, Hai Shu, Liming Zhong, Haipeng Shen, Hongtu Zhu. Automatic brain tumor seg-mentation with domain adaptation. 4th International MICCAI Brainlesion Workshop, BrainLes 2018 held in conjunction with the Medical Image Computing for Computer Assisted Intervention Conference, MIC-CAI 2018—Granada, Spain. 10.1007/978-3-030-11726-9_34

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (P C), a tablet P C, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 410, input device 412 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EP ROM), Electrically Erasable Programmable Read-Only Memory (EEP ROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the disclosure described herein.

It should be appreciated that any of the components or modules referred to with regards to any of the present disclosure embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Additional descriptions of aspects of the present disclosure will now be provided with reference to the accompanying drawings. The drawings form a part hereof and show, by way of illustration, specific embodiments or examples.

1. A computer implemented method of distinguishing tumor progression from treatment-related necrosis in digital images of a subject, the method comprising:
using a computer having a processor connected to computer memory storing software that, when executed, performs computer instruction steps of a machine learning architecture comprising:
collecting magnetic resonance image (MRI) data of a selected anatomical portion of the subject;
collecting dynamic positron emission tomography (dPET) data for the subject with a tracer applied to the anatomical portion of the subject;
co-registering MRI data frames with dPET data frames and storing a co-registered dPET volume of frames in the computer memory;
using the co-registered dPET volume, calculating and saving a tracer influx constant (Ki) map for the tracer at the anatomical portion of the subject;
segmenting respective tumor data from the MRI data and the Ki maps on a frame by frame basis;
applying segmented MRI data and segmented Ki maps to respective sections of a dual encoder convolutional neural network (CNN);
concatenating respective output latent feature vectors from the respective sections of the dual encoder; and
feeding concatenated feature vectors to fully connected layers of the machine learning architecture to distinguish tumor progression from treatment-related necrosis of the anatomical portion.

2. The method of claim 1, further comprising injecting the tracer into the subject to model glucose transport to the anatomical portion of the subject.

3. The method of claim 1, further comprising injecting Fluorine-18 fluorodeoxyglucose (18F-FDG) as a surrogate marker for glucose metabolism.

4. The method of claim 1, further comprising training the CNN utilizing a supervised transfer learning procedure.

5. The method of claim 1 further comprising storing three dimensional (3D) co-registered dPET tumor volumes in the computer memory.

6. A computer implemented method of distinguishing tumor progression from treatment-related necrosis in digital images of a subject, the method comprising:
using a computer having a processor connected to computer memory storing software that, when executed, performs computer instruction steps of a multimodal deep learning architecture comprising:
collecting magnetic resonance image (MRI) data of a selected anatomical portion of the subject;
collecting dynamic positron emission tomography (dPET) data for the subject with a tracer applied to the anatomical portion of the subject;
co-registering MRI data frames with dPET data frames and storing multi-channel parametric positron emission tomography (PET) volumes as three dimensional (3D) parametric PET maps in the computer memory;
using the 3D parametric PET maps to identify and store multi-modal image features in the computer memory;
segmenting respective tumor data from both the MRI data and the 3D parametric PET maps on a frame by frame basis;
applying segmented MRI data and selected multi-modal image features from the segmented 3D parametric PET maps to respective sections of at least one convolutional neural network (CNN);
concatenating respective output latent feature vectors from the respective sections of the at least one CNN; and
feeding concatenated feature vectors to fully connected layers of the multimodal architecture to distinguish tumor progression from treatment-related necrosis of the anatomical portion of the subject.

7. The method of claim 6, further comprising, prior to storing the 3D parametric PET maps, performing a step of calculating an image derived blood input function (IDIF) that identifies an amount of tracer in the blood available for the anatomical portion to use.

8. The method of claim 7, wherein calculating the IDIF comprises segmenting internal carotid arteries (ICA) of the subject from the 3D parametric PET maps co-registered with the MRI data frames.

9. The method of claim 8, wherein calculating the IDIF comprises correcting the IDIF with multi-parameter modeling correcting for partial volume (PV) effects and spill over (SP) contamination to store a model corrected blood input function (MCIF) in the computer memory.

10. The method of claim 9, further comprising feeding the MCIF and the dPET data for the subject into a graphical Patlak model that performs a voxel-wise linear regression on the data to derive a rate of tracer uptake, Ki, as a slope.

11. The method of claim 10, further comprising utilizing the MCIF to compute voxel by voxel parametric maps of tracer kinetic rate constants and tracer influx constant.

12. The method of claim 8, further comprising convolving an ICA segmentation to compute an average blood time-activity curve across all time frames to produce a tracer time activity curve as an initial value for the IDIF.

13. The method of claim 6, wherein the anatomical portion is a brain of a subject with a tumor therein, and the method further comprises collecting radiomics features from respective voxels of the 3D parametric PET maps and/or radiomics from corresponding images of MRI tumor volumes, wherein the radiomics features comprise at least one of first-order statistics, 2D and 3D shape-descriptors, or texture level features.

14. The method of claim 6, wherein concatenating respective output latent feature vectors further comprises adding to a concatenated feature vector with multimodal image features from the 3D parametric PET maps, wherein the multimodal features comprise metabolic uptake rate Ki, individual rate constants K1 to K3, total blood volume, tumor time-activity curves (TAC), or standardized uptake values (SUV).

15. The method of claim 6, wherein collecting magnetic resonance image (MRI) data further comprises collecting multi-channel MRI data comprising T1 image data, T1c image data, t2/FLAIR image data, perfusion imaging, and diffusor tensor imaging (DTI).

16. The method of claim 15, wherein concatenating respective output latent feature vectors further comprises adding to a concatenated feature vector with the multi-channel MRI data.

17. The method of claim 6, further comprising collecting static PET data for the anatomical portion of the subject and wherein concatenating respective output latent feature vectors further comprises the static PET data.

18. The method of claim 6, further comprising retrieving demographic data regarding the subject and wherein concatenating respective output latent feature vectors further comprises the demographic data.

19. A system comprising a computer having a processor connected to computer memory and in communication with an MRI imaging device and a PET scanning device, wherein the computer memory stores software that implements the multimodal deep learning architecture of claim 6.

20. The system of claim 19, wherein the anatomical feature of the subject is a brain having a tumor.
